SEQUENCE LISTING (32 sequences)

SEQ ID NO: 1 - protein, 118 aa, Artificial Sequence
  Synthetic Sequence
QVQLQQPGAELVKPGASVKLSCKASGYTFTSYWMHWVKQRPGRGLEWIGRIDPNSGGTKSNEKFNNKATLTVDKSSSTAYMQLSSLTSEDSAVYYCARWWDYDYFDYWGQGTTLTVSS

SEQ ID NO: 2 - protein, 8 aa, Artificial Sequence
  Synthetic Sequence
GYTFTSYW

SEQ ID NO: 3 - protein, 8 aa, Artificial Sequence
  Synthetic Sequence
IDPNSGGT

SEQ ID NO: 4 - protein, 11 aa, Artificial Sequence
  Synthetic Sequence
ARWWDYDYFDY

SEQ ID NO: 5 - protein, 113 aa, Artificial Sequence
  Synthetic Sequence
DIVMTQSPSSLTVTAGEKVTMSCKSSQSLLNSGNQKNYLTWYQQKPGQPPKLLIYWASTRESGVPDRFTGSGSGTDFTLTISNVQAEDLAVYYCQNDYNYPLTFGAGTKLELK

SEQ ID NO: 6 - protein, 12 aa, Artificial Sequence
  Synthetic Sequence
QSLLNSGNQKNY

SEQ ID NO: 7 - skipped

SEQ ID NO: 8 - protein, 9 aa, Artificial Sequence
  Synthetic Sequence
QNDYNYPLT

SEQ ID NO: 9-16 - skipped (8 entries)

SEQ ID NO: 17 - protein, 120 aa, Artificial Sequence
  Synthetic Sequence
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCARDGTTVVATLAYWGQGTLVTVSA

SEQ ID NO: 18 - protein, 9 aa, Artificial Sequence
  Synthetic Sequence
GYSITSGYY

SEQ ID NO: 19 - protein, 7 aa, Artificial Sequence
  Synthetic Sequence
ISYDGSN

SEQ ID NO: 20 - protein, 13 aa, Artificial Sequence
  Synthetic Sequence
ARDGTTVVATLAY

SEQ ID NO: 21 - protein, 106 aa, Artificial Sequence
  Synthetic Sequence
QIVLTQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELK

SEQ ID NO: 22 - protein, 5 aa, Artificial Sequence
  Synthetic Sequence
SSVSY

SEQ ID NO: 23 - skipped

SEQ ID NO: 24 - protein, 9 aa, Artificial Sequence
  Synthetic Sequence
QQYHSYPPT

SEQ ID NO: 25 - protein, 118 aa, Artificial Sequence
  Synthetic Sequence
QVQLQQPGAELVKPGTSVKLSCQASGYTFASYWMHWVKQRPGRGLEWIGRIDPNSGGTKYNEKFKSKVTLTVDKPSSAAYMQLSSLTSEDSAVYYCARWWDYDYFDYWGQGTTLTVSS

SEQ ID NO: 26 - protein, 11 aa, Artificial Sequence
  Synthetic Sequence
GYTFASYWSEQ

SEQ ID NO: 27 - protein, 8 aa, Artificial Sequence
  Synthetic Sequence
IDPNSGGT

SEQ ID NO: 28 - protein, 11 aa, Artificial Sequence
  Synthetic Sequence
ARWWDYDYFDY

SEQ ID NO: 29 - protein, 113 aa, Artificial Sequence
  Synthetic Sequence
DIVMTQSPSSLTVTAGEKVTMSCKSSQSLLNSGNQKRYLTWYQQKPGQPPKLLIYWASTRESGVPDRFTGSGSGTDFTLTISSVQAEDLAVYYCQNDYNYPLTFGSGTKLELK

SEQ ID NO: 30 - protein, 12 aa, Artificial Sequence
  Synthetic Sequence
QSLLNSGNQKRY

SEQ ID NO: 31 - skipped

SEQ ID NO: 32 - protein, 9 aa, Artificial Sequence
  Synthetic Sequence
QNDYNYPLT

(19) United States
(12) Patent Application Publication
(10) Pub. No.: US 2024/0043529 A1
(43) Pub. Date: Feb. 8, 2024

(54) METHODS AND COMPOSITIONS RELATED TO BCL2 AND BIM HETERODIMER ANTIBODIES

(71) Applicant: Eutropics Pharmaceuticals, Inc., Cambridge, MA (US)

(72) Inventors: Michael H. CARDONE, Dorchester, MA (US); Andrew KINLOCH, Cambridge, MA (US); Stephen GILLIES, Cambridge, MA (US)

(21) Appl. No.: 18/269,036

(22) PCT Filed: Dec. 22, 2021
(86) PCT No.: PCT/US21/64882

§ 371 Date: Jun. 22, 2023

Related U.S. Application Data
(60) Provisional application No. 63/128,933, filed on Dec. 22, 2020.

(51) Int. Cl. C07K 16/28; C12N 15/63; G01N 33/68

(52) U.S. Cl. CPC C07K 16/28; C12N 15/63; G01N 33/6893; C07K 2317/565; C07K 2317/567; G01N 2800/52

(57) ABSTRACT

The present disclosure relates to compositions and methods of determining cancer cell sensitivity to treatment using antibodies that detect heterodimers comprising Bc1-2 proteins selected from Bc12 and BIM. The disclosure also provides methods for predicting a cancer patient's sensitivity to the cancer treatment.

### CROSS-REFERENCE TO RELATED APPLICATIONS

This application claims priority to U.S. Provisional Application No. 63/128,933, filed on Dec. 22, 2020, the entire contents of which are incorporated herein.

### DESCRIPTION OF THE TEXT FILE SUBMITTED ELECTRONICALLY

The instant application contains a Sequence Listing which has been submitted in ASCII format via EFS-Web and is hereby incorporated by reference in its entirety. Said ASCII copy, created about Dec. 20, 2021, is named “EUTR-022PC_105444-5022_Sequence_Listing_ST25.txt” and is about 12,288 bytes in size.

### BACKGROUND

Treatment of cancer patients continues to be challenging due to the heterogeneity of tumors, and the absence of well-defined molecular targets. As more therapies are approved for different types of cancer, predictive and prognostic biomarkers, together with targeted cancer therapies, are likely to improve drug efficacy and provide a better roadmap for clinical decision making, so that these therapies can be directed to patients who are most likely to benefit. Unfortunately, the biomarkers available for cancer therapy are not sufficient.

One hallmark for tumors is the dysregulation of apoptotic pathways, which play an important role in tumorigenesis. Moreover, resistance to apoptosis is associated with desensitization to conventional cytotoxic and targeted therapies. Thus, the induction of cell death by targeting apoptotic pathways has been an attractive therapeutic strategy.

Two apoptotic pathways are well known. The extrinsic pathway is activated by ligand engagement of cell surface death receptors, and the intrinsic pathway initiates apoptosis by involving a wide-array of non-receptor-mediated stimuli that produce intracellular signals that act directly on targets within the cell and are typically mitochondrial-initiated events.

Several chemotherapeutic agents are known to cause apoptosis. The mechanism often involves changes in the levels and interactions of a key protein family of the intrinsic pathway: the BCL-2 family of proteins. The members of the BCL-2 family are typically designated based on their BCL-2 homology (BH) domains and involvement in apoptosis regulation. Traditionally, these proteins are organized into one of three subfamilies: (1) anti-apoptotic, and (2) BH3-only (pro-apoptotic), or (3) pore-forming or ‘executioner’ (pro-apoptotic) proteins.

To understand the molecular mechanics of individual apoptotic proteins, BH3 profiling is a functional assay that is used to measure tumor cell mitochondrial priming. BH3 profiling measures mitochondrial outer membrane permeabilization (MOMP) following exposure to a peptide-mimicking BH3 domains of BH3-only proteins. MOMP is measured indirectly by the fluorescent dye JC-1, which measures potential across the mitochondrial inner membrane. This potential from the JC-1 dye rapidly degrades in response to MOMP.

However, despite its use for BH3 profiling, the JC-1 readout has several difficulties, which include the inconsistent fluorescent signaling measurements. Further, direct measurement of the protein levels of individual BH3-only proteins, instead of a functional signal, is confounded by the fact that changes in these levels are not consistently correlated with sensitivity to the test anti-cancer agents being tested.

Additionally, combining the functional BH3 measurement with direct measurement of the protein levels of individual BH3-only proteins is complicated and not suited for solid tumor or fixed specimens.

Thus, there is a need for new compositions and methods that provide improved predictive testing for cancer treatment, so that drugs are assigned to patients who are most likely to benefit.

### SUMMARY

Accordingly, the present disclosure is based, in part, on the discovery of several antibodies that each specifically bind to a Bc12 and BIM heterodimer. The disclosure further provides antibodies are useful for detecting a Bc12 and BIM heterodimer in a solid tumor or liquid tumor sample from a patient, or a blood cancer sample from a patient, and determining a ratio of the heterodimer to a reference value, the ratio being predictive of a patient's sensitivity to the cancer treatment. As such, the disclosed antibodies provide improved compositions and methods predictive testing for cancer treatment.

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYTFTSYW (SEQ ID NO: 2), or a variant thereof, the heavy chain CDR2 sequence is IDPNSGGT (SEQ ID NO: 3), or a variant thereof, and the heavy chain CDR3 sequence is ARWWDYDYFDY (SEQ ID NO: 4), or a variant thereof, and (ii) a light chain variable region comprising light chain CDR1, CDR2, and CDR3 sequences, wherein the light chain CDR1 sequence is QSLLNSGNQKNY (SEQ ID NO: 6), or a variant thereof, the light chain CDR2 sequence is WAS (SEQ ID NO: 7), or a variant thereof, and the light chain CDR3 sequence is QNDYNYPLT (SEQ ID NO: 8), or a variant thereof

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYSITSGYY (SEQ ID NO: 18), or a variant thereof, the heavy chain CDR2 sequence is ISYDGSN (SEQ ID NO: 19), or a variant thereof, and the heavy chain CDR3 sequence is ARDGTTVVATLAY (SEQ ID NO: 20), or a variant thereof, and (ii) a light chain variable region comprising light chain CDR1, CDR2, and CDR3 sequences, wherein the light chain CDR1 sequence is SSVSY (SEQ ID NO: 22), or a variant thereof, the light chain CDR2 sequence is RTS (SEQ ID NO: 23), or a variant thereof, and the light chain CDR3 sequence is QQYHSYPPT (SEQ ID NO: 24), or a variant thereof.

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYTFASYW(SEQ ID NO: 26), or a variant thereof, the heavy chain CDR2 sequence is IDPNSGGT (SEQ ID NO: 27), or a variant thereof, and the heavy chain CDR3 sequence is ARWWDYDYFDY (SEQ ID NO: 28), or a variant thereof, and (ii) a light chain variable region comprising light chain CDR1, CDR2, and CDR3 sequences, wherein the light chain CDR1 sequence is QSLLNSGNQKRY (SEQ ID NO: 30), or a variant thereof, the light chain CDR2 sequence is WAS (SEQ ID NO: 31), or a variant thereof, and the light chain CDR3 sequence is QNDYNYPLT (SEQ ID NO: 32), or a variant thereof.

In some embodiments, the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In some embodiments, the variable region FW sequences are human. In some embodiments, the antibody or antibody format, or fragment thereof, comprises a human heavy chain and light chain constant regions. In some embodiments, the constant regions are selected from the group consisting of human IgG1, IgG2, IgG3, and IgG4.

In some embodiments, the antibody or antibody format, or fragment thereof, comprises: (i) a heavy chain variable region sequence comprising the amino acid sequence set forth in SEQ ID NO: 1, or the amino acid sequence of SEQ ID NO: 1 having at least about 90% identity thereto; and (ii) a light chain variable region sequence comprising the amino acid sequence of SEQ ID NO: 5, or the amino acid sequence of SEQ ID NO: 5 having at least about 90% identity thereto. In some embodiments, the antibody or antibody format, or fragment thereof, comprises an amino acid sequence having at least about 95%, or 97%, or 98% identity with SEQ ID NO: 1 and/or SEQ ID NO. 5.

In some embodiments, the antibody or antibody format, or fragment thereof, comprises: (i) a heavy chain variable region sequence comprising the amino acid sequence set forth in SEQ ID NO: 17, or the amino acid sequence of SEQ ID NO: 17 having at least about 90% identity thereto; and (ii) a light chain variable region sequence comprising the amino acid sequence of SEQ ID NO: 21, or the amino acid sequence of SEQ ID NO: 21 having at least about 90% identity thereto. In some embodiments, the antibody or antibody format, or fragment thereof, comprises an amino acid sequence having at least about 95%, or 97%, or 98% identity with SEQ ID NO: 17 and/or SEQ ID NO. 21.

In some embodiments, the antibody or antibody format, or fragment thereof, comprises: (i) a heavy chain variable region sequence comprising the amino acid sequence set forth in SEQ ID NO: 25, or the amino acid sequence of SEQ ID NO: 25 having at least about 90% identity thereto; and (ii) a light chain variable region sequence comprising the amino acid sequence of SEQ ID NO: 29, or the amino acid sequence of SEQ ID NO: 29 having at least about 90% identity thereto. In some embodiments, the antibody or antibody format, or fragment thereof, comprises an amino acid sequence having at least about 95%, or 97%, or 98% identity with SEQ ID NO: 25 and/or SEQ ID NO. 29.

In some embodiments, disclosed herein is a polynucleotide comprising a nucleic acid sequence encoding the antibody or antibody format, or fragment thereof. In some embodiments, disclosed herein is a vector comprising the polynucleotide. In some embodiments, the present disclosure provides a host cell comprising the vector.

In various embodiments, disclosed herein is a pharmaceutical composition comprising the antibody or antibody format, or fragment thereof, and a pharmaceutically acceptable excipient.

In some embodiments, the present disclosure provides a method for predicting a patient's sensitivity or response to a cancer treatment, comprising: (a) contacting a sample with an antibody or antibody format, or fragment thereof, as described herein, wherein the antibody recognizes a heterodimer comprising two B-cell lymphoma 2 (BCL-2) proteins selected from Bc12 and BIM, the sample being a specimen from a solid tumor or liquid tumor sample of the patient; (b) detecting a signal that indicates the amount of the heterodimer; and (c) determining a ratio of the amount of heterodimer present in the sample from step (b) to a reference value, wherein the reference value comprises the amount of one of the Bc12 and BIM monomers of the heterodimer in the sample, the ratio being predictive of the patient's sensitivity to the cancer treatment.

In some embodiments, the present disclosure provides a method for predicting a patient's sensitivity or response to a cancer treatment, comprising: (a) contacting a sample with an antibody or antibody format, or fragment thereof, as described herein, wherein the antibody recognizes a heterodimer comprising two B-cell lymphoma 2 (BCL-2) proteins selected from Bc12 and BIM, and an antibody or antibody format, or fragment thereof, that recognizes one of the Bc12 and BIM protein monomers of the heterodimer, the sample being a specimen from a solid tumor or liquid tumor of the patient; (b) detecting a signal that indicates the amount of the heterodimer and a signal that indicates the amount of the monomer; and (c) determining a ratio based on the amount heterodimer to the amount of the monomer, the ratio being predictive of the patient's sensitivity to the cancer treatment.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DESCRIPTION OF THE DRAWINGS

FIG. 1 shows three line graphs of antigen reactivity of supernatants from three hybridomas “2H3”, “10C10” and “28A9”, each expressing mouse IgG specific for the Bc12:Bim heterodimeric complex.

FIG. 2 is a display of two graphs analogous to those in FIG. 1, demonstrating HSB2B hybridoma supernatant (clones “10C10” and “2H3”, as indicated) specific reactivity for the Bc12:Bim complex, when tested amongst a more exhaustive range of antigens, including the Bc12 family member Mcl1.

FIG. 3 is a bar graph demonstrating the capacity of the HSB2B mAbs to react with a physiologically relevant Bc12:Bim heterodimer complex formed using the full-length Bim protein.

FIG. 4 demonstrates how, by immunohistochemistry (IHC) on fixed formalin paraffin embedded (FFPE) tissue, HSB2B clones “2H3” and “10C10” can react with small cell lung cancer tissue.

FIG. 5 shows IHC on FFPE small cell lung cancer tissue demonstrating that each of the present engineered heterodimer specific antibodies (HSAs) can have reactivity with small cell lung cancer.

FIG. 6 is IHC on FFPE breast cancer tissue demonstrating that the present engineered HSAs can have different levels of reactivity with breast cancer biopsies from different patients. The present anti-BC12/BIM antibodies are labelled “HSB2B” and a clone number (see Table A); “HSBXB” is an antibody against Bc1-x1/Bim and “HSMCB” is an antibody against MCL-1/BIM.

FIG. 7 is a bar graph demonstrating that each of the present HSAs can be used on flow cytometry. Cell-lines EOL-1, KG1a and U2932 (previously demonstrated to have high levels of the Bc12:Bim heterodimer) were stained with the indicated HSAs and detected with an Alexa Fluor 647 conjugated anti-mouse IgG. The present anti-BC12/BIM antibodies are labelled “HSB2B” and a clone number (see Table A); “HSBXB” is an antibody against Bc1-x1/Bim and “HSMCB” is an antibody against MCL-1/BIM.

FIG. 8 shows HSB2B reactivity with different Bc12 and Bc1_XL heterodimers. The present anti-BC12/BIM antibodies are labelled “HSB2B” and a clone number (see Table A).

### DETAILED DESCRIPTION

The present disclosure is based, in part, on the discovery of compositions and methods for detecting whether a patient is sensitive to a cancer treatment for instance, by an antibody that specifically binds to a Bc12/BIM heterodimer. The disclosure further provides compositions and methods that are useful for detecting a heterodimer comprising two B-cell lymphoma 2 (BCL-2) proteins selected from Bc12 and BIM in a solid tumor or liquid tumor sample from a patient, or a blood cancer sample from a patient, and determining a ratio of the heterodimer to a reference value, the ratio being predictive of a patient's sensitivity to the cancer treatment. Importantly, the present methods give information about a cancer patient response based on a direct signal, as opposed to a functional one.

Apoptosis is a process of programmed cell death mediated by a number of signaling pathways that converge at the mitochondria. A group of mitochondrial proteins, i.e., the B cell leukemia/lymphoma-2 (BCL-2) family of proteins, regulates this process. More specifically, pro-apoptotic and anti-apoptotic BCL-2 proteins form heterodimers with their cognate regulating BCL-2 proteins (i.e., the BH3-only BCL-2 proteins), thereby effecting cell death or survival signals.

One of the hallmarks of apoptosis is mitochondrial outer membrane permeabilization (MOMP), a process regulated by the Bc1-2 family of proteins. The activity of this family of proteins is linked to the onset of lymphoid and several solid tumor cancers and is believed in many cancers to be a key mediator of resistance to chemotherapy. Bc1-2 proteins are regulated by distinct protein-protein interactions between pro-survival (anti-apoptotic) and pro-apoptotic members. These interactions occur primarily through BH3 (Bc1-2 homology domain-3) mediated binding. Apoptosis-initiating signaling occurs for the most part upstream of the mitochondria and causes the translocation of short, BH3-only, Bc1-2 family members to the mitochondria where they either activate or sensitize MOMP. The activator BH3 only proteins, Bim and Bid, bind to and directly activate the effector, pro-apoptotic proteins Bax and Bak, and also bind to and inhibit the anti-apoptotic Bc1-2 family proteins, Bc1-2, Mc1-1, Bfl-1, Bc1-w and Bc1-xL. The sensitizer BH3 proteins, Bad, Bik, Noxa, Hrk, Bmf and Puma, bind only to the anti-apoptotic Bc1-2 family proteins, Bc1-2, Mc1-1, Bfl-1, Bc1-w and Bc1-xL, blocking their anti-apoptotic functions. Without wishing to be bound by theory, each sensitizer protein has a unique specificity profile. For example, Noxa (A and B) bind with high affinity to Mc1-1, Bad binds to Bc1-xL and Bc1-2 but only weakly to Mc1-1, and Puma binds well to all three targets. An anti-apoptotic function of these proteins is the sequestering of the activator BH3 protein Bim and Bid by binding to form heterodimers. Displacement of these activators by sensitizer peptides or treatments results in Bax/Bak-mediated apoptotic commitment. These interactions can have various outcomes, including, without limitation, homeostasis, cell death, sensitization to apoptosis, and blockade of apoptosis.

Most effective cancer drugs induce apoptosis in target cancer cells. However, one significant shortfall in current cancer treatment is that different cancer cells can respond to an apoptosis-inducing drug in a variety of manners. This is due, in part, to the presence of different heterodimers between the pro/anti-apoptotic BCL-2 proteins and the regulatory BH3-only BCL-2 proteins in those cancer cells.

In some aspects, the present disclosure provides a method for predicting a patient's sensitivity or response to a cancer treatment, comprising contacting a sample with an antibody or antibody format that recognizes a heterodimer comprising two B-cell lymphoma 2 (BCL-2) proteins selected from Bc12 and BIM, the sample being a specimen from a solid tumor or liquid tumor of the patient; or a blood cancer sample from a patient, detecting a signal that indicates the amount of the heterodimer; and determining a ratio based on the amount of heterodimer present in the sample to a reference value, wherein the reference value comprises the amount of one of the Bc12 and BIM protein monomers of the heterodimer in the sample, the ratio being predictive of a patient's sensitivity to the cancer treatment. In some embodiments, the detected signal is from the Bc12/BIM heterodimeric antibody; for example, detecting a signal indicates the amount of the heterodimer, wherein the signal is from the Bc12/BIM heterodimeric antibody.

In another aspect, the present disclosure provides a method for predicting a patient's sensitivity or response to a cancer treatment, comprising: contacting a sample with an antibody or antibody format that recognizes a heterodimer comprising two B-cell lymphoma 2 (BCL-2) proteins selected from Bc12 and BIM and an antibody or antibody format that recognizes one of the Bc12 and BIM protein monomers of the heterodimer, the sample being a specimen from a solid tumor or liquid tumor of the patient; or a blood cancer sample from a patient, detecting a signal that indicates the amount of the heterodimer and the amount of the monomer; and determining a ratio based on the amount heterodimer to the amount of the monomer, the ratio being predictive of a solid tumor, liquid tumor or a blood cancer patient's sensitivity to the cancer treatment. In some embodiments, the detected signal is from the Bc12/BIM heterodimeric antibody; for example, detecting a signal indicates the amount of the heterodimer, wherein the signal is from the Bc12/BIM heterodimeric antibody.

Generation and Production of Antibodies that Bind to Bc12 and BIM Heterodimer and their Utility in Cancer

The present disclosure can use the determination of a cancer cell's predisposition to undergo apoptosis to elucidate the cancer's susceptibility to a particular treatment. One way this can be done is by using the disclosed antibodies that bind to a Bc12 and Bim heterodimer which regulate apoptosis. Formation of a heterodimer induces conformational changes in both members of the heterodimer, resulting in exposure of antigenic epitopes that are sequestered in both members before dimerization. The isolated antibodies of the present disclosure specifically recognize such an epitope and only bind to a Bc12 and Bim heterodimer, and not to either non-dimerized member.

One aspect of this disclosure features an isolated antibody that specifically binds to a Bc12 and BIM heterodimer. The Bc1-2 family includes both Bc1-2 proteins (monomers, i.e., Bc12 and BIM), and naturally-occurring heterodimers formed of Bc12 and BIM proteins. The heterodimer contains a first Bc1-2 protein selected from Bim and a second Bc1-2 protein selected from Bc12. In some embodiments, the Bc12 protein in the Bc12 and Bim heterodimer is associated with Bim, or is associated with the BH3 domain peptide derived from Bim. In some embodiments, the Bc12 protein in the Bc12 and BIM heterodimer is associated with multiple different BH3 only proteins, or BH3 peptides derived from them. For example, in some embodiments, the Bc12 protein in the Bc12 and Bim heterodimer is associated with an activator BH3 protein, and the activator BH3 protein is selected from BID and BIM. In some embodiments, the Bc12 protein is associated with a sensitizer BH3 protein. The sensitizer BH3 protein is selected from BAD, BIK, NOXA A, NOXA B, HRK, BMF, and PUMA. In some embodiments, the Bc12 protein is associated a multidomain pro-apoptotic protein, and the multidomain pro-apoptotic protein is selected from BAX and BAK.

The methods of the present disclosure also provide a ratio of the Bc12 and BIM heterodimer to one of a Bc12 and BIM monomer.

The compositions of the present disclosure include an antibody or antibody format, or fragment thereof, that recognizes a Bc12/Bim heterodimer (HSB2B). In various embodiments, the antibody is a full-length multimeric protein that includes two heavy chains and two light chains. Each heavy chain includes one variable region (e.g., VH) and at least three constant regions (e.g., CH₁, CH₂ and CH₃), and each light chain includes one variable region (VL) and one constant region (CL). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the antibody or antibody format, or fragment thereof, is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-Jun; 3(3): 310-317.

In various embodiments, the antibody or antibody format, or fragment thereof, is a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH₂ and CH₃).

In some embodiments, the antibody or antibody format, or fragment thereof is selected from one or more of a monoclonal antibody, polyclonal antibody, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody.

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYTFTSYW (SEQ ID NO: 2), or a variant thereof, the heavy chain CDR2 sequence is IDPNSGGT (SEQ ID NO: 3), or a variant thereof, and the heavy chain CDR3 sequence is ARWWDYDYFDY (SEQ ID NO: 4), or a variant thereof, and (ii) a light chain variable region comprising light chain CDR1, CDR2, and CDR3 sequences, wherein the light chain CDR1 sequence is QSLLNSGNQKNY (SEQ ID NO: 6), or a variant thereof, the light chain CDR2 sequence is WAS (SEQ ID NO: 7), or a variant thereof, and the light chain CDR3 sequence is QNDYNYPLT (SEQ ID NO: 8), or a variant thereof

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYSITSGYY (SEQ ID NO: 18), or a variant thereof, the heavy chain CDR2 sequence is ISYDGSN (SEQ ID NO: 19), or a variant thereof, and the heavy chain CDR3 sequence is ARDGTTVVATLAY (SEQ ID NO: 20), or a variant thereof, and (ii) a light chain variable region comprising light chain CDR1, CDR2, and CDR3 sequences, wherein the light chain CDR1 sequence is SSVSY (SEQ ID NO: 22), or a variant thereof, the light chain CDR2 sequence is RTS (SEQ ID NO: 23), or a variant thereof, and the light chain CDR3 sequence is QQYHSYPPT (SEQ ID NO: 24), or a variant thereof.

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is GYTFASYW(SEQ ID NO: 26), or a variant thereof, the heavy chain CDR2 sequence is IDPNSGGT (SEQ ID NO: 27), or a variant thereof, and the heavy chain CDR3 sequence is ARWWDYDYFDY (SEQ ID NO: 28), or a variant thereof, and (ii) a light chain variable region comprising light chain CDR1, CDR2, and CDR3 sequences, wherein the light chain CDR1 sequence is QSLLNSGNQKRY (SEQ ID NO: 30), or a variant thereof, the light chain CDR2 sequence is WAS (SEQ ID NO: 31), or a variant thereof, and the light chain CDR3 sequence is QNDYNYPLT (SEQ ID NO: 32), or a variant thereof.

In some embodiments, the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In some embodiments, the variable region FW sequences are human. In some embodiments, the antibody or antibody format, or fragment thereof, comprises a human heavy chain and light chain constant regions. In some embodiments, the constant regions are selected from the group consisting of human IgG1, IgG2, IgG3, and IgG4.

In some embodiments, the antibody or antibody format, or fragment thereof, comprises: (i) a heavy chain variable region sequence comprising the amino acid sequence set forth in SEQ ID NO: 1, or the amino acid sequence of SEQ ID NO: 1 having at least about 90% identity thereto; and (ii) a light chain variable region sequence comprising the amino acid sequence of SEQ ID NO: 5, or the amino acid sequence of SEQ ID NO: 5 having at least about 90% identity thereto. In some embodiments, the antibody or antibody format, or fragment thereof, comprises an amino acid sequence having at least about 95%, or 97%, or 98% identity with SEQ ID NO: 1 and/or SEQ ID NO. 5.

In some embodiments, the antibody or antibody format, or fragment thereof, comprises: (i) a heavy chain variable region sequence comprising the amino acid sequence set forth in SEQ ID NO: 17, or the amino acid sequence of SEQ ID NO: 17 having at least about 90% identity thereto; and (ii) a light chain variable region sequence comprising the amino acid sequence of SEQ ID NO: 21, or the amino acid sequence of SEQ ID NO: 21 having at least about 90% identity thereto. In some embodiments, the antibody or antibody format, or fragment thereof, comprises an amino acid sequence having at least about 95%, or 97%, or 98% identity with SEQ ID NO: 17 and/or SEQ ID NO. 21.

In some embodiments, the antibody or antibody format, or fragment thereof, comprises: (i) a heavy chain variable region sequence comprising the amino acid sequence set forth in SEQ ID NO: 25, or the amino acid sequence of SEQ ID NO: 25 having at least about 90% identity thereto; and (ii) a light chain variable region sequence comprising the amino acid sequence of SEQ ID NO: 29, or the amino acid sequence of SEQ ID NO: 29 having at least about 90% identity thereto. In some embodiments, the antibody or antibody format, or fragment thereof, comprises an amino acid sequence having at least about 95%, or 97%, or 98% identity with SEQ ID NO: 25 and/or SEQ ID NO. 29.

In various embodiments, the antibody or antibody format, or fragment thereof, or variant thereof, may comprise an amino acid sequence having one or more amino acid mutations (e.g., substitutions or deletions) relative to any of the sequences disclosed herein. In some embodiments, the one or more amino acid mutations may be independently selected from substitutions, insertions, deletions, and truncations. In embodiments, the antibody or antibody format, or fragment thereof, comprises a sequence that has about 1, 2, 3, 4, 5, 6, 7, 8, 9, or 10 or more amino acid mutations with respect to any one of the amino acid sequences disclosed herein.

In embodiments, the present compositions comprise one or more sequences of Table A, or a variant thereof (e.g. having one of more amino acid mutations).

TABLE A

| | | Hv | Hv | Hv | | Lv | Lv | Lv |
|---|---|---|---|---|---|---|---|---|
| Clone | Hv | CDR1 | CDR2 | CDR3 | Lv | CDR1 | CDR2 | CDR3 |
| 10C10F7 | QVQLQQPGAELVKP | GYTFT | IDPNSG | ARWW | DIVMTQSPSSLTVTAG | QSLLN | WAS | QND |
| | GASVKLSCKASGYT | SYW | GT (SEQ | DYDYF | EKVTMSCKSSQSLLN | SGNQ | (SEQ | YNYP |
| | FTSYWMHWVKQRP | (SEQ | ID NO: | DY | SGNQKNYLTWYQQK | KNY | ID NO: | LT |
| | GRGLEWIGRIDPNS | ID NO: | 3) | (SEQ | PGQPPKLLIYWASTR | (SEQ | 7) | (SEQ |
| | GGTKSNEKFNNKAT | 2) | | ID NO: | ESGVPDRFTGSGSGT | ID NO: | | ID NO: |
| | LTVDKSSSTAYMQL | | | 4) | DFTLTISNVQAEDLAV | 6) | | 8) |
| | SSLTSEDSAVYYCA | | | | YYCQNDYNYPLTFGA | | | |
| | RWWDYDYFDYWG | | | | GTKLELK (SEQ ID | | | |
| | QGTTLTVSS (SEQ | | | | NO: 5) | | | |
| | ID NO: 1) | | | | | | | |
| 28A9D11 | DVQLQESGPGLVKP | GYSIT | ISYDGS | ARDG | QIVLTQSPAIMSASPG | SSVSY | RTS | QQY |
| | SQSLSLTCSVTGYSI | SGYY | N (SEQ | TTVVA | EKVTISCSASSSVSYM | (SEQ | (SEQ | HSYP |
| | TSGYYWNWIRQFP | (SEQ | ID NO: | TLAY | YWYQQKPGSSPKPWI | ID NO: | ID NO: | PT |
| | GNKLEWMGYISYD | ID NO: | 19) | (SEQ | YRTSNLASGVPARFS | 22) | 23 | (SEQ |
| | GSNNYNPSLKNRISI | 18) | | ID NO: | GSGSGTSYSLTISSM | | | ID NO: |
| | TRDTSKNQFFLKLN | | | 20) | EAEDAATYYCQQYHS | | | 24) |
| | SVTTEDTATYYCAR | | | | YPPTFGAGTKLELK | | | |
| | DGTTVVATLAYWG | | | | (SEQ ID NO: 21) | | | |
| | QGTLVTVSA (SEQ | | | | | | | |
| | ID NO: 17) | | | | | | | |
| 2H3G7 | QVQLQQPGAELVKP | GYTFA | IDPNSG | ARWW | DIVMTQSPSSLTVTAG | QSLLN | WAS | QND |
| | GTSVKLSCQASGYT | SYW | GT (SEQ | DYDYF | EKVTMSCKSSQSLLN | SGNQ | (SEQ | YNYP |
| | FASYWMHWVKQRP | (SEQ ID | ID NO: | DY | SGNQKRYLTWYQQK | KRY | ID NO: | LT |
| | GRGLEWIGRIDPNS | NO: 26) | 27) | (SEQ | PGQPPKLLIYWASTR | (SEQ | 31) | (SEQ |
| | GGTKYNEKFKSKVT | | | ID NO: | ESGVPDRFTGSGSGT | ID NO: | | ID NO: |
| | LTVDKPSSAAYMQL | | | 28) | DFTLTISSVQAEDLAV | 30) | | 32) |
| | SSLTSEDSAVYYCA | | | | YYCQNDYNYPLTFGS | | | |
| | RWWDYDYFDYWG | | | | GTKLELK (SEQ ID | | | |
| | QGTTLTVSS (SEQ | | | | NO: 29) | | | |
| | ID NO: 25) | | | | | | | |

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

Bc1-2 proteins, which are present in mitochondria, are major regulators of the commitment to programmed cell death and executioners of death/survival signals. (See, e.g., Reed, Natural Clinical Practice Oncology, 3:388-398 (2006), Green et al., Cancer Cell 1:19-30 (2002), and Adams et al., Cold Spring Harb. Symp. Quant. Biol. 70:469-477 (2005)) There are four sub-groups of Bc1-2 proteins: (i) multi-domain anti-apoptotic Bc1-2 proteins, (ii) multi-domain pro-apoptotic Bc1-2 proteins, (iii) activator BH3-only Bc1-2 proteins, and (iv) sensitizer BH3-only Bc1-2 proteins. Table 1 below lists major human Bc1-2 proteins and their GenBank accession numbers:

TABLE 1
Human Bcl-2 Proteins

| Bcl-2 | Bcl-2 | Gene Bank |
|---|---|---|
| Proteins Class | proteins | Accession numbers |
| Multi Domain | Bcl-2 | AAH27258(Jul. 15, 2006( |
| Anti-Apoptotic | Mcl-1 | AAF64255 (Jul. 15, 2000) |
| | BCL-XL | AAH19307 (Jul. 15, 2015) |
| | BFL-1 | Q16548 (Mar. 3, 2009) |
| | BFL-W | AAB09055 (Sep. 29, 1996) |
| Multi Domain | BAX | Q07812 (Apr. 14, 2009) |
| Pro-Apoptotic | BAK | Q16611 (Apr. 14, 2009) |
| Activator | BID | P55957 (Mar. 3, 2009) |
| BH3 only class | BIM | O43521 (Apr. 14, 2009) |
| Sensitisor | BAD | CAG30276 (Jun. 29, 2004) |
| BH3 only class | NOXA | Q13794 (Mar. 3, 2009) |
| | PUMA. | Q9BXH1 (Apr. 14, 2009) |
| | HRK, | AAC34931 (Sep. 9, 1998) |
| | Mule | Q7Z67Z (Apr. 14, 2009) |
| | BIK | CAG30276 (Oct. 16, 2008) |
| | BMF | AAH63928 (Aug. 19^{th}, 2004) |

Other Bc1-2 proteins, if any, can be identified by a homologous search using the amino acid sequence of a known Bc1-2 protein as a query.

Polypeptides can be identified based on homology to the BH3 domain, and polypeptides can possess at least about 50%, about 60%, about 70%, about 80%, about 90%, about 95%, about 96%, about 97%, about 98%, about 99% sequence homology to the amino acid sequences of the polypeptides disclosed in Table 1. Preferred variants are those that have conservative amino acid substitutions made at one or more predicted non-essential amino acid residues. For example, a “conservative amino acid substitution” is one in which the amino acid residue is replaced with an amino acid residue having a similar side chain. In a further embodiment, the BH3 domain peptide is an activator or a sensitizer of apoptosis. In a preferred embodiment, the BH3 domain peptide is a sensitizer.

If a cell is pre-set to undergo drug-induced apoptosis (e.g. the cell is dependent on Bc1-2 polypeptide activity for survival), the Bc12 and BIM heterodimer antibodies of the present disclosure can be used to identify the specific Bc12 and BIM proteins that are responsible for apoptotic block.

It is known that members in one subgroup of Bc1-2 proteins form heterodimers with members in a different subgroup to regulate apoptosis. Formation of a heterodimer induces conformational changes in both members of the heterodimer, resulting in exposure of antigenic epitopes that are sequestered in both members before dimerization. The isolated Bc12 and BIM heterodimer antibodies of the present disclosure specifically recognize such an epitope. In other words, the antibodies disclosed herein can specifically bind to a heterodimer of the Bc1-2 family selected from Bc12 and BIM.

Briefly, without wishing to be bound by theory, as a result of aberrant phenotypes, cancer cells develop blocks in apoptosis pathways. These blocks make cancer cells both resistant to some therapies, and, surprisingly, make some cancer cells sensitive to other therapies. The concept of “oncogene addiction” describes the phenomena of the acquired dependence of cancer cells on, or addiction to, particular proteins for survival. Cancer cells can be, but are not always, pre-set to undergo apoptosis and this is a function of these cells being dependent on any, or all of the anti-apoptotic Bc1-2 family proteins for their otherwise unintended survival. This provides insight into the likelihood of a cancer cell to respond to treatment.

Cancer cells, without wishing to be bound by theory, exhibit abnormalities, such as DNA damage, genetic instability, abnormal growth factor signaling, and abnormal or missing matrix interactions, any of which should typically induce apoptosis through the intrinsic (mitochondrial) apoptosis pathway. However, rather than respond to these apoptosis signals cancer cells survive. Often, in doing so, these cells become highly dependent on selected blocks to chronic apoptosis signals. This adaptation provides a survival mechanism for the cancer cells; however, these adaptations can also make cancer cells susceptible to particular apoptosis inducing therapies. A crucial event that commits a cell to die by intrinsic apoptosis is the permeabilization of the mitochondrial outer membrane (MOMP) and the release of molecules that activate the effector caspases. In many cases, MOMP is the point of no return in the intrinsic apoptosis pathway. The Bc1-2 family proteins are the key regulators of MOMP, and their activity is linked to the onset of lymphoid and several solid tumor cancers, and is believed in many cancers to be the key mediator of resistance to chemotherapy.

Bc1-2 proteins are regulated by distinct protein-protein interactions between pro-survival (anti-apoptotic) and pro-apoptotic members. These interactions occur primarily through BH3 (Bc1-2 homology domain-3) mediated binding. Apoptosis-initiating signaling occurs for the most part upstream of the mitochondria and causes the translocation of short, BH3-only, Bc1-2 family members to the mitochondria where they either activate or sensitize MOMP. The activator BH3 only proteins, Bim and Bid, bind to and directly activate the effector, pro-apoptotic proteins Bax and Bak, and also bind to and inhibit the anti-apoptotic Bc1-2 family proteins, Bc1-2, Mc1-1, Bfl-1, Bc1-w and Bc1-xL. The sensitizer BH3 proteins, Bad, Bik, Noxa, Hrk, Bmf and Puma, bind only to the anti-apoptotic Bc1-2 family proteins, Bc1-2, Mc1-1, Bfl-1, Bc1-w and Bc1-xL, blocking their anti-apoptotic functions. Without wishing to be bound by theory, each sensitizer protein has a unique specificity profile. For example, Noxa (A and B) bind with high affinity to Mc1-1, Bad binds to Bc1-xL and Bc1-2 but only weakly to Mc1-1, and Puma binds well to all three targets. An anti-apoptotic function of these proteins is the sequestering of the activator BH3 protein Bim and Bid. Displacement of these activators by sensitizer peptides results in Bax/Bak-mediated apoptotic commitment. These interactions can have various outcomes, including, without limitation, homeostasis, cell death, sensitization to apoptosis, and blockade of apoptosis.

A defining feature of cancer cells in which apoptotic signaling is blocked is an accumulation of the BH3 only activator proteins at the mitochondrial surface, a result of these proteins being sequestered by the anti-apoptotic proteins. This accumulation and proximity to their effector target proteins accounts for increased sensitivity to antagonism of Bc1-2 family proteins in the “BH3 primed” state.

In some embodiments, a cell yielding a high apoptotic response to Noxa (A or B) is Bc12 primed. In some embodiments, Puma reflects pan-Bc1-2 family priming. In this way, cells that are dependent on either Bc12 or Bim, or both proteins, or on several Bc1-2 family members are readily distinguished so that appropriate treatment may be tailored accordingly. The distinctions in mitochondrial response to these peptides guides the use of therapies that are known to work through pathways that funnel into either Bc12 or Bim affected intrinsic signaling. The use of a Bc12 inhibiting compound may be indicated in such cases. In some embodiments, the present methods also indicate or contraindicate therapies that target entities upstream of Bc12 or Bim.

The antibodies of the present disclosure can be a whole immunoglobulin or a fragment thereof that retains antigen-binding activity. In some embodiments, the antibodies of the present disclosure can be a genetically modified immunoglobulin, including scFv antibody, chimeric antibody, or a humanized antibody. In some embodiments, the antibody or antibody format is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody. In some embodiments, the antibody or antibody format further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. In some embodiments, the variable region FW sequences are human. The antibody or antibody format can further comprise a human heavy chain and light chain constant regions. In some embodiments, the constant regions are selected from the group consisting of human IgG1, IgG2, IgG3, and IgG4. The term “isolated antibody,” as used herein, refers to an antibody substantially free from naturally associated molecules, i.e., the naturally associated molecules constituting at most 20% by dry weight of a preparation containing the antibody.

The antibodies of the present disclosure may be prepared by conventional methods. (See, e.g., Harlow and Lane, (1988) Antibodies: A Laboratory Manual, Cold Spring Harbor Laboratory, New York) For example, a synthetic heterodimer of the Bc1-2 family may be prepared by producing two members of the heterodimer protein separately using a section of a key binding motif, followed by synthesizing the key epitope and inducing a portion of one member of the heterodimer, the ligand, and the full-length protein of the other member of the heterodimer, the receptor. The functionality of the synthetic heterodimer can be checked using in vitro binding assays. Once determined, the binding fidelity is maintained in the synthetic heterodimer, and then the ligand portion can be modified to contain a benzoyl phenylalanine (Anaspec, Fremont, CA, USA) in place of one of several potential aromatic amino acids. Each protein fragment can be further tested for binding fidelity as detailed above. Once selected, the binding ligand can be covalently attached by exposure to activating exposure to UV light at 450 nM for up to 8 hours. The synthetic heterodimer can then be purified by FPLC and be used as an immunogen for injection into a mouse host.

To produce antibodies that bind to the heterodimer, the heterodimer may be optionally coupled to a carrier protein (e.g., KLH) and mixed with an adjuvant, followed by injection into a host animal. Antibodies produced in the animal can then be purified by heterodimer affinity chromatography. Commonly employed host animals include rabbits, mice, guinea pigs, and rats. Various adjuvants may be used to increase the immunological response, which depends on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum. Polyclonal antibodies, i.e., heterogeneous populations of antibody molecules, are present in the sera of the immunized animal.

Monoclonal antibodies, i.e., homogeneous populations of antibody molecules, are prepared using standard hybridoma technology. (See, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.)) In particular, monoclonal antibodies may be obtained by any technique that provides for the production of antibody molecules by continuous cell lines in culture. (See, Kohler et al. (1975) Nature 256, 495; Kosbor et al. (1983) Immunol Today 4, 72; Cole et al. (1983) Proc. Natl. Acad. Sci. USA 80, 2026, and the EBV-hybridoma technique (Cole et al. (1983); see also Monoclonal Antibodies and Cancer Therapy, Alan R. Liss, Inc., pp. 77-96)) Such antibodies can be of any immunoglobulin class including IgG, IgM, IgE, IgA, IgD, and any subclass thereof. The hybridoma producing the monoclonal antibodies of the present disclosure may be cultivated in vitro or in vivo. The ability to produce high titers of monoclonal antibodies in vivo makes it a particularly useful method of production.

In addition, techniques developed for the production of “chimeric antibodies” can be used. (See, e.g., Morrison et al. (1984) Proc. Natl. Acad. Sci. USA 81, 6851; Neuberger et al. (1984) Nature 312, 604; and Takeda et al. (1984) Nature 314:452) A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage or yeast library of scFv antibodies. scFv antibodies are formed by linking the heavy and light chain fragments of the Fv region via an amino acid bridge.

Moreover, antibody fragments can be generated by known techniques. For example, such fragments include, but are not limited to, F(ab′)2 fragments that can be produced by pepsin digestion of an antibody molecule, and Fab fragments that can be generated by reducing the disulfide bridges of F(ab′) 2 fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the present disclosure (see, e.g., Green et al. (1994) Nature Genetics 7, 13; and U.S. Pat. Nos. 5,545,806 and 5,569,825).

In embodiments, the antibodies prepared by any of the methods described above may be confirmed for their binding to a Bc1-2 heterodimer selected from Bc12 and BIM. In embodiments, the antibodies may be subjected to a negative selection to exclude those that also were bound to a non-dimerized member of the heterodimer. In these experiments, negative selection is the unbound Bc12 protein or the unbound BIM protein. For example, each of the two members, i.e., monomer A and monomer B, may be labeled with a distinct fluorescent dye, i.e., dye x and dye y, respectively. Dyes x and y have different optimal emission wavelengths. The antibody was first incubated with labeled monomer A, labeled monomer B, or the AB heterodimer (double labeled) for a suitable period and then captured by GamaBind Sepharose beads. Whether the antibody is capable of binding to either monomer or to the heterodimer is determined based on the fluorescent signal released from the captured antibody. Antibodies that bound to the heterodimer and not to either non-dimerized member are selected.

The antibodies, in embodiments, may be confirmed for their binding to a Bc1-2 heterodimer selected from Bc12 and BIM using an ELISA assay.

The antibodies disclosed herein can be used in a method of detecting the presence or absence of a Bc1-2 heterodimer in a sample obtained from a subject (e.g., a patient), particularly, a fixed tissue sample or a mitochondrial fraction, by conventional methods, e.g., immunohistochemistry (IHC) staining. For example, an antibody that recognizes a heterodimer comprising two B-cell lymphoma 2 (BCL-2) proteins selected from Bc12 and BIM may be used to profile the presence of Bc12 and BIM on the outer membranes of mitochondria in multiple tissue samples from a patient. Tissues at various disease stages (e.g., malignancy stages) can be collected from the same patient. Mitochondrial fractions can be prepared from these tissues and, using a plurality of the antibodies of the present disclosure, the fractions can be profiled for the presence/absence of Bc1-2 heterodimers selected from Bc12 and BIM.

Also disclosed herein is a method of predicting a human patient's sensitivity or responsiveness to a drug that interferes with formation of a particular Bc1-2 heterodimer selected from Bc12 and BIM, directly or indirectly, based on the presence of the Bc12 and BIM heterodimer in the patient.

It is well known that Bc1-2 proteins play an essential role in regulating apoptosis via formation of heterodimers between members in different Bc1-2 sub-groups. See Table 1 above. An activator BH3-only Bc1-2 protein (i.e., BID or BIM) binds to a multi-domain pro-apoptotic Bc1-2 protein (i.e., BAX or BAK), triggering mitochondrial outer membrane permeabilization (MOMP), which leads to cell death. A multi-domain anti-apoptotic Bc1-2 protein (e.g., Bc1-2) can bind to BAX and BAK, and also sequester an activator BH3-only protein from binding to BAX or BAK. Consequently, it blocks the MOMP process, resulting in cell survival. The activity of a multi-domain anti-apoptotic Bc1-2 protein is regulated by the sensitizer BH3-only proteins. This subgroup of Bc1-2 proteins promotes apoptosis by binding to the anti-apoptotic Bc1-2 protein, displacing the activator BH3-only Bc1-2 proteins so that they are released to bind to the pro-apoptotic Bc1-2 proteins, thereby triggering the MOMP process. In short, there are two types of Bc1-2 heterodimers: (1) pro-apoptotic Bc1-2 heterodimers, formed between an activator BH3-only Bc1-2 protein and a multi-domain pro-apoptotic Bc1-2 protein or between a sensitizer BH3-only Bc1-2 protein and a multi-domain anti-apoptotic Bc1-2 protein; and (2) anti-apoptotic Bc1-2 heterodimers, formed between a multi-domain anti-apoptotic Bc1-2 protein and an activator BH3-only Bc1-2 protein or between a multi-domain anti-apoptotic Bc1-2 protein and a multi-domain pro-apoptotic Bc1-2 protein. Formation of pro-apoptotic Bc1-2 heterodimers promotes apoptosis while formation of anti-apoptotic heterodimers promotes cell survival.

The presence of a particular pro- or anti-apoptotic Bc1-2 heterodimer in a subject (e.g., a patient) is known to indicate that patient's responsiveness to a drug that blocks formation of the particular heterodimer and inhibits its function. (See, e.g., Delbridge and Strasser A. Cell Death Differ. 2015 July; 22(7):1071-80. doi: 10.1038/cdd.2015.50)

In some embodiments of the present disclosure, the drug is a mimetic of a BH3-only protein that competes against the BH3-only protein for binding to its cognate partner. In other embodiments, the drug targets an upstream apoptotic factor and ultimately blocks formation of a Bc1-2 heterodimer.

Many cancer drugs induce apoptosis in cancer cells by blocking formation of anti-apoptotic Bc1-2 heterodimers. The presence of a particular anti-apoptotic Bc1-2 heterodimer selected from in a cancer patient indicates that this patient is sensitive to a drug that interferes with formation of this anti-apoptotic Bc1-2 heterodimer. (See Robert et al., Clinical Pharmacology and Therapeutics 101; 1, January 2017). On the other hand, apoptosis inhibitors can be used for treating neurodegenerative disease or cardiovascular disease, both of which involve apoptosis. In this context, the presence of a particular pro-apoptotic Bc1-2 heterodimer in a neurodegenerative disease patient or a cardiovascular disease patient, for example, indicates that such a patient is sensitive to an apoptosis inhibitor that blocks formation of the particular pro-apoptotic Bc1-2 heterodimer.

In some embodiments, the sensitivity is characterized by (a) the presence of apoptosis in the sample; (b) the presence of an anti-apoptotic Bc1-2 heterodimer selected from Bc12 and BIM in the sample, indicating the patient is sensitive to a drug that interferes with formation of the heterodimer; (c) genetic risk factors; family history; personal history; race and ethnicity; features of the certain tissues; various benign conditions (e.g. nonproliferative lesions); previous chest radiation; carcinogen exposure and the like.

In some embodiments, the method does not involve a functional readout of mitochondrial outer membrane permeabilization (MOMP). In some embodiments, the method does not involve a dye-based detection of cell membrane potential.

Illustrative Clinical Decisions

In some embodiments, the methods described herein are useful in the evaluation of a solid tumor or liquid tumor sample from a patient, or a blood cancer sample from a patient, for example, for evaluating diagnosis, prognosis, and response to treatment. In various aspects, the present disclosure comprises evaluating a solid tumor, liquid tumor, or a blood cancer sample from a patient. In various embodiments, the evaluation may be selected from diagnosis, prognosis, and response to treatment.

In various aspects, the methods of the present disclosure may be used to treat a cancer patient. For example, the methods may further comprise administering a cancer treatment to the patient if the ratio is predictive of sensitivity to the cancer treatment. In some embodiments, the methods may further comprise treating the patient with a reduced dose or less frequent and/or shortened regimen of the cancer treatment if the ratio is predictive of sensitivity to the cancer treatment. In some embodiments, the methods may further comprise treating the patient with an increased dose or more frequent and/or prolonged regimen of the cancer treatment if the ratio is predictive of sensitivity to the cancer treatment. In some embodiments, the methods may further comprise withholding cancer treatment to the patient if the ratio is predictive of a lack of sensitivity to the cancer treatment. In some embodiments, the methods may further comprise treating the patient with a different cancer treatment if the ratio is predictive of a lack of sensitivity to the cancer treatment.

For instance, in various embodiments, the sample presents a ratio of more dimer than monomer. For instance, the ratio of dimer to monomer may be about 20:1 or about 15:1, or about 10:1, or about 9:1, or about 8:1, or about 7:1, or about 6:1, or about 5:1, or about 4:1, or about 3:1, or about 2:1. In various embodiments, the sample presents a ratio of more monomer than dimer. For instance, the ratio of monomer to dimer may be about 20:1 or about 15:1, or about 10:1, or about 9:1, or about 8:1, or about 7:1, or about 6:1, or about 5:1, or about 4:1, or about 3:1, or about 2:1. In various embodiments, the ratio of dimer to monomer is equivalent (i.e. about 1:1).

Diagnosis refers to the process of attempting to determine or identify a possible disease or disorder, such as, for example, cancer. Prognosis refers to predicting a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, and progression free survival, time to progression, and probability of recurrence.

In various embodiments, the present methods direct a clinical decision regarding whether a patient is to receive a specific treatment. In one embodiment, the present methods are predictive of a positive response to neoadjuvant and/or adjuvant chemotherapy or a non-responsiveness to neoadjuvant and/or adjuvant chemotherapy. In one embodiment, the present methods are predictive of a positive response to a pro-apoptotic agent or an agent that operates via apoptosis and/or an agent that does not operate via apoptosis or a non-responsiveness to apoptotic effector agent and/or an agent that does not operate via apoptosis. In various embodiments, the present disclosure directs the treatment of a cancer patient, including, for example, what type of treatment should be administered or withheld.

In some embodiments, the present methods direct a cancer treatment regarding one or more of anti-cancer drugs, chemotherapy, antagonist of an anti-apoptotic protein, surgery, adjuvant therapy, and neoadjuvant therapy.

In one embodiment, the present methods direct a clinical decision regarding whether a patient is to receive adjuvant therapy after primary, main or initial treatment, including, without limitation, a single sole adjuvant therapy. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease.

In some embodiments, the present methods direct a patient's treatment to include adjuvant therapy. For example, a patient that is scored to be responsive to a specific treatment may receive such treatment as adjuvant therapy. Further, the present methods may direct the identity of an adjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as adjuvant therapy. Accordingly, in some embodiments, the present methods provide for providing or withholding adjuvant therapy according to a patient's likely response. In this way, a patient's quality of life, and the cost of care, may be improved.

In various embodiments, the present methods direct a clinical decision regarding whether a patient is to receive neoadjuvant therapy, e.g. therapy to shrink and/or downgrade the tumor prior to surgery. In some embodiments, neoadjuvant therapy means chemotherapy administered to cancer patients prior to surgery. In some embodiments, neoadjuvant therapy means an agent, including those described herein, administered to cancer patients prior to surgery. Types of cancers for which neoadjuvant chemotherapy is commonly considered include, for example, breast, colorectal, ovarian, cervical, bladder, and lung.

In some embodiments, the present methods direct a patient's treatment to include neoadjuvant therapy. For example, a patient that is scored to be responsive to a specific treatment may receive such treatment as neoadjuvant therapy. Further, the present methods may direct the identity of a neoadjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as neoadjuvant therapy. Accordingly, in some embodiments, the present methods provide for providing or withholding neoadjuvant therapy according to a patient's likely response. In this way, a patient's quality of life, and the cost of case, may be improved.

In some embodiments, the present methods direct a clinical decision regarding whether a patient is to receive a specific type of treatment (e.g., one or more of anti-cancer drugs, chemotherapy, antagonist of an anti-apoptotic protein, surgery, adjuvant therapy, and neoadjuvant therapy). In some embodiments, the cancer treatment is one or more of a SMAC mimetic, BH3 mimetic, proteasome inhibitor, histone deacetylase inhibitor, glucocorticoid, steroid, monoclonal antibody, antibody-drug conjugate, or thalidomide derivative. In some embodiments, the present methods are a guiding test for patient treatment.

In some embodiments, the present methods comprise a cancer treatment and the cancer treatment is a checkpoint inhibitor. The checkpoint inhibitor can be an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. The agent that targets PD-1 can be an antibody or antibody format specific for PD-1, optionally selected from nivolumab, pembrolizumab, and pidilizumab. The agent that targets PD-L1 can be an antibody or antibody format specific for PD-L1, optionally selected from atezolizumab, avelumab, durvalumab, and BMS-936559. The agent that targets CTLA-4 can be an antibody or antibody format specific for CTLA-4, optionally selected from ipilimumab and tremelimumab.

In some embodiments, the present methods provide information about the likely response that a patient is to have to a particular treatment. In some embodiments, the present methods provide a high likelihood of response and may direct treatment, including aggressive treatment. In some embodiments, the present methods provide a low likelihood of response and may direct cessation of treatment, including aggressive treatment, and the use of palliative care, to avoid unnecessary toxicity from ineffective chemotherapies for a better quality of life.

In an illustrative embodiment, the present method will indicate a likelihood of response to a specific treatment. For example, in some embodiments, the present methods indicate a high or low likelihood of response to a pro-apoptotic agent and/or an agent that operates via apoptosis and/or an agent that operates via apoptosis driven by direct protein modulation. In various embodiments, illustrative pro-apoptotic agents and/or agents that operate via apoptosis and/or an agent that operates via apoptosis driven by direct protein modulation include ABT-263 (Navitoclax), and obatoclax, WEP, bortezomib, Venetoclax (ABT-199), and carfilzomib. In some embodiments, the present methods indicate a high or low likelihood of response to an agent that does not operate via apoptosis and/or an agent that does not operate via apoptosis driven by direct protein modulation. In various embodiments, illustrative agents that do not operate via apoptosis include kinesin spindle protein inhibitors, cyclin-dependent kinase inhibitor, Arsenic Trioxide (TRISENOX), MEK inhibitors, pomolidomide, azacytidine, decitibine, vorinostat, entinostat, dinaciclib, gemtuzumab, BTK inhibitors, PI3 kinase delta inhibitors, lenolidimide, anthracyclines, cytarabine, melphalam, Aky inhibitors, mTOR inhibitors.

In an illustrative embodiment, the present method will indicate whether a patient is to receive a pro-apoptotic agent or an agent that operates via apoptosis for cancer treatment. In another illustrative embodiment, the present method will indicate whether a patient is to receive an agent that does not operate via apoptosis.

In a specific embodiment, the present methods are useful in predicting a cancer patient's response to any of the treatments (including agents) described herein.

In various embodiments, a cancer treatment is administered or withheld based on the methods described herein. Illustrative treatments include surgical resection, radiation therapy (including the use of the compounds as described herein as, or in combination with, radiosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics).

Illustrative Treatments

In illustrative embodiments, the disclosure selects a treatment agent. Examples of such agents include, but are not limited to, one or more of anti-cancer drugs, chemotherapy, surgery, adjuvant therapy, and neoadjuvant therapy. In one embodiment, the cancer treatment is one or more of a BH3 mimetic, epigenetic modifying agent, topoisomerase inhibitor, cyclin-dependent kinase inhibitor, and kinesin-spindle protein stabilizing agent. In some embodiments, the BH3 mimetic is selected from ABT-737 and ABT-263 (navitoclax), Bc1-2 specific Venetoclax (Venclexta, ABT-199), MCL-1 specific 563845 and AMG176 and ADZ5991, BCL-XL specific A-1155463 and A1331852, BFL-1/MCL-1 specific EU5346 or combinations thereof. In another embodiment, the cancer treatment is a proteasome inhibitor; and/or a modulator of cell cycle regulation (by way of non-limiting example, a cyclin dependent kinase inhibitor); and/or a modulator of cellular epigenetic mechanistic (by way of non-limiting example, one or more of a histone deacetylase (HDAC) (e.g. one or more of vorinostat or entinostat), azacytidine, decitabine); and/or an anthracycline or anthracenedione (by way of non-limiting example, one or more of epirubicin, doxorubicin, mitoxantrone, daunorubicin, idarubicin); and/or a platinum-based therapeutic (by way of non-limiting example, one or more of carboplatin, cisplatin, and oxaliplatin); cytarabine or a cytarabine-based chemotherapy; a BH3 mimetic (by way of non-limiting example, one or more of BCL2, BCLXL, or MCL1); an inhibitor of BIM, and an inhibitor of Bc12. In some embodiments, the cancer treatment blocks formation of the particular heterodimer detected. In some embodiments, the cancer treatment perturbs or reduces formation of the particular heterodimer detected.

In various embodiments, the cancer treatment comprises one or more chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, Erbitux, Avastin, Pertuzumab, anti-CD20 antibodies, Rituxan, ocrelizumab, ofatumumab, DXL625, HERCEPTIN®, or any combination thereof.

In various embodiments, the disclosure pertains to cancer treatments including, without limitation, one or more of alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′, 2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation, dacogen, velcade, and pharmaceutically acceptable salts, acids or derivatives of any of the above.

Illustrative Detection Methods

In various embodiments, the present methods comprise predicting a patient's sensitivity to a cancer treatment. In some embodiments, the detection of the heterodimer employs an immunohistochemistry (IHC), flow cytometry, or immunofluorescent method.

In various embodiments, the methods involve evaluating a presence, absence, or level of a protein and/or a nucleic acid. In various embodiments, the present methods comprise evaluating a presence, absence, or level of a protein and/or a nucleic acid which can enhance the specificity and/or sensitivity of a Bc12 and BIM heterodimer ratio. In some embodiments, the evaluating is of a marker for patient response. In some embodiments, the present methods comprise measurement using one or more of immunohistochemical staining (i.e., IHC), western blotting, in cell western, immunofluorescent staining, ELISA, and fluorescent activating cell sorting (FACS), or any other method described herein or known in the art. The present methods may comprise contacting an antibody with a tumor specimen (e.g. biopsy or tissue or body fluid) to identify an epitope that is specific to the tissue or body fluid and that is indicative of a state of a cancer.

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, N Y, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, CA).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In another embodiment, the measurement comprises evaluating a presence, absence, or level of a nucleic acid. A person skilled in the art will appreciate that a number of methods can be used to detect or quantify the DNA/RNA levels of appropriate markers.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP. A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses.

Illustrative Cancers and Patients

In some embodiments the disclosure provides a method for determining a cancer treatment and/or comprises a patient's tumor or cancer cell specimen. A cancer or tumor refers to an uncontrolled growth of cells and/or abnormal increased cell survival and/or inhibition of apoptosis which interferes with the normal functioning of the bodily organs and systems. A subject that has a cancer or a tumor is a subject having objectively measurable cancer cells present in the subject's body. Included in this disclosure are benign and malignant cancers, as well as dormant tumors or micrometastases. Cancers which migrate from their original location and seed vital organs can eventually lead to the death of the subject through the functional deterioration of the affected organs.

In various embodiments, the disclosure is applicable to pre-metastatic cancer, or metastatic cancer. Metastasis refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are often detected through the sole or combined use of magnetic resonance imaging (MRI) scans, computed tomography (CT) scans, blood and platelet counts, liver function studies, chest X-rays and bone scans in addition to the monitoring of specific symptoms.

The methods described herein are directed toward the prognosis of cancer, diagnosis of cancer, treatment of cancer, and/or the diagnosis, prognosis, treatment, prevention or amelioration of growth, progression, and/or metastases of malignancies and proliferative disorders associated with increased cell survival, or the inhibition of apoptosis. In some embodiments, the cancer is a solid tumor, including, but not limited to, non-small lung cell carcinoma, ovarian cancer, and melanoma.

In some embodiments, the sample is an infiltrating lymphocyte of the patient.

In some embodiments, the solid tumor is selected from lung cancer, breast cancer, prostate cancer, melanoma, pancreatic cancer, kidney cancer, colon cancer, and ovarian cancer. In some embodiments, the lung cancer is selected from non-small cell lung cancer (NSCLC) and small cell lung cancer (SCLC). In some embodiments, the breast cancer is triple negative breast cancer. In some embodiments, the prostate cancer is androgen independent prostate cancer.

In some embodiments, the sample is a liquid tumor, or a blood cancer. For example, hematopoietic cells are blood-forming cells in the body. Hematopoiesis (development of blood cells) begins in the bone marrow and depending on the cell type, further maturation occurs either in the periphery or in secondary lymphoid organs such as the spleen or lymph nodes. Hematopoietic disorders are recognized as clonal diseases, which are initiated by somatic and/or inherited mutations that cause dysregulated signaling in a progenitor cell. The wide range of possible mutations and accompanying signaling defects accounts for the diversity of disease phenotypes observed within this group of disorders. Hematopoietic disorders fall into three major categories: Myelodysplastic syndromes, myeloproliferative disorders, and acute leukemias. Examples of hematopoietic disorders include non-B lineage derived, such as acute myeloid leukemia (AML), Chronic Myeloid Leukemia (CIVIL), non-B cell acute lymphocytic leukemia (ALL), myelodysplastic disorders, myeloproliferative disorders, polycythemias, thrombocythemias, or non-B atypical immune lymphoproliferations. Examples of B-Cell or B cell lineage derived disorder include Chronic Lymphocytic Leukemia (CLL), B lymphocyte lineage leukemia, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia.

Acute myeloid leukemia (AML), myelodysplastic syndrome (MDS), and myeloproliferative neoplasms (MPN) are examples of distinct myeloid hematopoietic disorders. However, it is recognized that these disorders share clinical overlap in that 30% of patients with MDS and 5-10% of patients with MPN will go on to develop AML. Below are current descriptions of these myeloid disorders.

AML is characterized by an uncontrolled proliferation of immature progenitor cells of myeloid origin including, but not limited to, myeloid progenitor cells, myelomonocytic progenitor cells, and immature megakaryoblasts. It is becoming clear that AML is really a heterogeneous collection of neoplasms with elements of differing pathophysiology, genetics and prognosis. Under WHO guidelines, diagnosis of AML can be made when blasts (immature cells) are present at 20% or more in peripheral blood or bone marrow sampling.

In some embodiments, the disclosure relates to one or more of the following cancers: adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumors, cerebellar astrocytoma, cervical cancer, chronic myeloproliferative disorders, colon cancer, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, lip and oral cavity cancer, liposarcoma, liver cancer, lung cancer (e.g. non-small cell, small cell), medulloblastoma, melanoma, Merkel cell carcinoma, mesothelioma, metastatic squamous neck cancer, mouth cancer, multiple endocrine neoplasia syndrome, mycosis fungoides, myelodysplastic syndromes, myelodysplastic/myeloproliferative diseases, myeloproliferative disorders, chronic, nasal cavity and paranasal sinus cancer, nasopharyngeal carcinoma, neuroblastoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, pleuropulmonary blastoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, testicular cancer, throat cancerm, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, and Wilms tumor.

The term subject, as used herein unless otherwise defined, is a mammal, e.g., a human, mouse, rat, hamster, guinea pig, dog, cat, horse, cow, goat, sheep, pig, or non-human primate, such as a monkey, chimpanzee, or baboon. The terms “subject” and “patient” are used interchangeably.

Illustrative Specimens

In some embodiments, the present disclosure includes the measurement of a tumor specimen, including biopsy or surgical specimen samples. In some embodiments, the specimen is selected from a frozen tumor tissue specimen, cultured cells, circulating tumor cells, and a formalin-fixed paraffin-embedded tumor tissue specimen. In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen.

In some embodiments, the sample is selected from a tumor biopsy, tissue biopsy, tumor resection, frozen tumor tissue specimen, lymph node, bone marrow, circulating tumor cells, cultured cells, a formalin-fixed paraffin embedded tumor tissue specimen, bronchoalveolar lavage, skin, hair, urine, and combinations thereof. In some embodiments, the tumor biopsy is selected from a core biopsy, needle biopsy, surgical biopsy, and an excisional biopsy.

In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C. to about −30° C. The specimen is embedded in a gel like medium consisting of, for example, poly ethylene glycol and polyvinyl alcohol. The frozen tissue is cut frozen with the microtome portion of the cryostat, and the section is optionally picked up on a glass slide and stained.

In some embodiments, the tumor specimen may be a biopsy sample, such as cultured cells. These cells may be processed using the usual cell culture techniques that are known in the art. These cells may be circulating tumor cells.

In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue.

In certain embodiments, the tumor specimen (or biopsy) contains less than 100 mg of tissue, or in certain embodiments, contains about 50 mg of tissue or less. The tumor specimen (or biopsy) may contain from about 20 mg to about 50 mg of tissue, such as about 35 mg of tissue.

The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In certain embodiments, the specimen is a human tumor-derived cell line. In certain embodiments, the specimen is a cancer stem cell. In other embodiments, the specimen is derived from the biopsy of a solid tumor, such as, for example, a biopsy of a colorectal, breast, prostate, lung, pancreatic, renal, or ovarian primary tumor.

In certain embodiments, the specimen is of epithelial origin. In some embodiments, the epithelial specimen is enriched by selection from a biopsy sample with an anti-epithelial cell adhesion molecule (EpCAM) or other epithelial cell binding antibody bound to solid matrix or bead.

In certain embodiments, the specimen is of mesenchymal origin. In some embodiments, the mesenchymal specimen is enriched by selection from a biopsy sample with a neural cell adhesion molecule (N-CAM) or neuropilin or other mesenchymal cell binding antibody bound to a solid matrix or bead.

In some embodiments, the specimen is derived from a circulating tumor cell.

Illustrative Clinical Factors and Additional Biomarkers

In some embodiments, the disclosure comprises determining one or more clinical factors of the patient. The disclosure can comprise detecting a heterodimer comprising two B-cell lymphoma 2 (BCL-2) proteins selected from Bc12 and BIM in a solid tumor or liquid tumor sample from a patient, and determining a ratio of the heterodimer and/or clinical factors to assess a patient response or predict a patient's sensitivity to cancer treatment. In some embodiments, the clinical factor comprises further classifying the patient for likelihood of clinical response to the cancer treatment based on one or more clinical factors of the patient. In some embodiments, the clinical factor comprises comparing the prediction of the patient's sensitivity to the cancer treatment with the likelihood of clinical response to the cancer treatment based on one or more clinical factors of the patient. In some embodiments, a clinical factor that provides patient response information in combination with the ratio study may not be linked to apoptosis. In some embodiments, a clinical factor is non-apoptosis affecting.

In some embodiments, the clinical factor is one or more of age, cytogenetic status, performance, histological subclass, gender, and disease stage. In some embodiments, the clinical factor further comprises measuring an additional biomarker selected from mutational status, single nucleotide polymorphisms, steady state protein levels, and dynamic protein levels.

In one embodiment, the clinical factor is age. In one embodiment, the patient age profile is classified as over about 10, or over about 20, or over about 30, or over about 40, or over about 50, or over about 60, or over about 70, or over about 80 years old.

In one embodiment, the clinical factor is cytogenetic status. In some cancers, such as Wilms tumor and retinoblastoma, for example, gene deletion or inactivation are responsible for initiating cancer progression, as chromosomal regions associated with tumor suppressors are commonly deleted or mutated. For example, deletions, inversions, and translocations are commonly detected in chromosome region 9p21 in gliomas, non-small-cell lung cancers, leukemia's, and melanomas. Without wishing to be bound by theory, these chromosomal changes may inactivate the tumor suppressor cyclin-dependent kinase inhibitor 2A. Along with these deletions of specific genes, large portions of chromosomes can also be lost. For instance, chromosomes 1p and 16q are commonly lost in solid tumor cells. Gene duplications and increases in gene copy numbers can also contribute to cancer and can be detected with transcriptional analysis or copy number variation arrays. For example, the chromosomal region 12q13-q14 is amplified in many sarcomas. This chromosomal region encodes a binding protein called MDM2, which is known to bind to a tumor suppressor called p53. When MDM2 is amplified, it prevents p53 from regulating cell growth, which can result in tumor formation. Further, certain breast cancers are associated with overexpression and increases in copy number of the ERBB2 gene, which codes for human epidermal growth factor receptor 2. Also, gains in chromosomal number, such as chromosomes 1q and 3q, are also associated with increased cancer risk.

Cytogenetic status can be measured in a variety of manners known in the art. For example, FISH, traditional karyotyping, and virtual karyotyping (e.g. comparative genomic hybridization arrays, CGH and single nucleotide polymorphism arrays) may be used. For example, FISH may be used to assess chromosome rearrangement at specific loci and these phenomena are associated with disease risk status. In some embodiments, the cytogenetic status is favorable, intermediate, or unfavorable.

In one embodiment, the clinical factor is performance. Performance status can be quantified using any system and methods for scoring a patient's performance status are known in the art. The measure is often used to determine whether a patient can receive chemotherapy, adjustment of dose adjustment, and to determine intensity of palliative care. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry. Higher performance status (e.g., at least 80%, or at least 70% using the Karnofsky scoring system) may indicate treatment to prevent progression of the disease state, and enhance the patient's ability to accept chemotherapy and/or radiation treatment. For example, in these embodiments, the patient is ambulatory and capable of self-care. In other embodiments, the evaluation is indicative of a patient with a low performance status (e.g., less than 50%, less than 30%, or less than 20% using the Karnofsky scoring system), so as to allow conventional radiotherapy and/or chemotherapy to be tolerated. In these embodiments, the patient is largely confined to bed or chair and is disabled even for self-care.

The Karnofsky score runs from 100 to 0, where 100 is “perfect” health and 0 is death. The score may be employed at intervals of 10, where: 100% is normal, no complaints, no signs of disease; 90% is capable of normal activity, few symptoms or signs of disease, 80% is normal activity with some difficulty, some symptoms or signs; 70% is caring for self, not capable of normal activity or work; 60% is requiring some help, can take care of most personal requirements; 50% requires help often, requires frequent medical care; 40% is disabled, requires special care and help; 30% is severely disabled, hospital admission indicated but no risk of death; 20% is very ill, urgently requiring admission, requires supportive measures or treatment; and 10% is moribund, rapidly progressive fatal disease processes.

The Zubrod scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all self-care but unable to carry out any work activities, up and about more than 50% of waking hours; 3, capable of only limited self-care, confined to bed or chair more than 50% of waking hours; 4, completely disabled, cannot carry on any self-care, totally confined to bed or chair; 5, dead.

In one embodiment, the clinical factor is histological subclass. In some embodiments, histological samples of tumors are graded according to Elston & Ellis, Histopathology, 1991, 19:403-10, the contents of which are hereby incorporated by reference in their entirety.

In one embodiment, the clinical factor is gender. In one embodiment, the gender is male. In another embodiment the gender is female.

In one embodiment, the clinical factor is disease stage. By way of non-limiting example, using the overall stage grouping, Stage I cancers are localized to one part of the body; Stage II cancers are locally advanced, as are Stage III cancers. Whether a cancer is designated as Stage II or Stage III can depend on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body.

In another embodiment, the method further comprises a measurement of an additional biomarker selected from mutational status, single nucleotide polymorphisms, steady state protein levels, and dynamic protein levels. In another embodiment, the method further comprises predicting a clinical response in the patient. In another embodiment, the clinical response is about 1, about 2, about 3, or about 5-year progression/event-free survival.

A variety of clinical factors have been identified, such as age profile and performance status. A number of static measurements of diagnosis have also been utilized, such as cytogenetics and molecular events including, without limitation, mutations in the genes MLL, AML/ETO, Flt3-ITD, NPM1 (NPMc+), CEBPα, IDH1, IDH2, RUNX1, ras, and WT1 and in the epigenetic modifying genes TET2 and ASXL, as well as changes in the cell signaling protein profile.

In some embodiments, the preventive methods comprise administering a treatment to a patient that is likely to be afflicted by cancer as guided by the methods described herein. In some embodiments, a subject is likely to be afflicted by cancer if the subject is characterized by one or more of a high risk for a cancer, a genetic predisposition to a cancer (e.g. genetic risk factors), a previous episode of a cancer (e.g. new cancers and/or recurrence), a family history of a cancer, exposure to a cancer-inducing agent (e.g. an environmental agent), and pharmacogenomics information (the effect of genotype on the pharmacokinetic, pharmacodynamic or efficacy profile of a therapeutic).

In some embodiments, a subject is likely to be afflicted by cancer if the subject is characterized by a high risk for a cancer. In some embodiments, a subject is likely to be afflicted by cancer if the subject is characterized by a genetic predisposition to a cancer. In some embodiments, a genetic predisposition to a cancer is a genetic clinical factor, as is known in the art. Such clinical factors may include, by way of example, MLH1, MSH2, MSH6, PMS1, PMS2 for at least colon, uterine, small bowel, stomach, urinary tract cancers. In some embodiments, a subject is likely to be afflicted by cancer if the subject is characterized by a previous episode of a cancer. In some embodiments, the subject has been afflicted with 1, or 2, or 3, or 4, or 5, or 6, previous episodes of cancer. In some embodiments, a subject is likely to be afflicted by cancer if the subject is characterized by a family history of a cancer. In some embodiments, a parent and/or grandparent and/or sibling and/or aunt/uncle and/or great aunt/great uncle, and/or cousin has been or is afflicted with a cancer. In some embodiments, a subject is likely to be afflicted by cancer if the subject is characterized by exposure to a cancer-inducing agent (e.g. an environmental agent). For example, exposing skin to strong sunlight is a clinical factor for skin cancer. By way of example, smoking is a clinical factor for cancers of the lung, mouth, larynx, bladder, kidney, and several other organs.

Further, in some embodiments, the any one of the following clinical factors may be useful in the methods described herein: gender; genetic risk factors; family history; personal history; race and ethnicity; features of the certain tissues; various benign conditions (e.g. non-proliferative lesions); previous chest radiation; carcinogen exposure and the like.

Further still, in some embodiments, the any one of the following clinical factors may be useful in the methods described herein: one or more of a cell surface marker CD33, a cell surface marker CD34, a FLT3 mutation status, a p53 mutation status, a phosphorylation state of MEK-1 kinase, and phosphorylation of serine at position 70 of Bc1-2.

In some embodiments, the clinical factor is expression levels of the cytokines, including, without limitation, interleukin-6. In some embodiments, interleukin-6 levels will correlate with likelihood of response in MINI patients, including a poor patient prognosis or a good patient prognosis.

In some embodiments, the likelihood of response is determined by assessing a percent priming. In certain embodiments, the priming is defined by the following equation:

$$\% Priming = [ 100 * ( \frac{negative control AUC - {Peptide}_{1} AUC}{negative control AUC - Positive {Control}_{avg} AUC} ) ] {Peptide}_{1} + [ 100 * ( \frac{negative control AUC - {Peptide}_{1} AUC}{negative control AUC - Positive {Control}_{avg} AUC} ) ] {Peptide}_{2} + … / ( n peptides )$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the negative control comprises a baseline negative control for either an area under a curve or a signal intensity;
the positive control comprises a baseline positive control for either an area under a curve or a signal intensity (e.g., any uncoupling agent); and
   the Peptide is one or more BH3 domain peptides, wherein (n) is normalized with the average number of replicates of the negative and positive controls.

In some embodiments, in combination with the preceding equation, the one or more clinical factors are selected to increase specificity and/or sensitivity of the BH3 profile for association with clinical response.

In some embodiments, the likelihood of response is determined by assessing a percent priming. In certain embodiments, the priming is defined by the following equation:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

In some embodiments, in combination with the preceding equation, the one or more clinical factors are selected to increase specificity and/or sensitivity of the BH3 profile for association with clinical response.

In some embodiments, the likelihood of clinical response is defined by a simplified form of the preceding equation, as shown here:

$$\% Priming = [ 100 * ( \frac{{DMSO}_{a v g} A UC - {Peptide}_{n} A U C}{{DMSO}_{a v g} A U C - C C C {P}_{a v g} A U C} ) ]$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

In some embodiments, in combination with the preceding equation, the one or more clinical factors are selected to increase specificity and/or sensitivity of the BH3 profile for association with clinical response.

In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS). In some embodiments, the signal intensity is a single time point measurement that occurs between about 5 min and about 300 min.

In some embodiments, the present disclosure provides a method for predicting a patient's responsiveness to a checkpoint inhibitor in a sample, comprising measuring the amount of a Bc12/BIM heterodimer, wherein the sample comprises an infiltrating lymphocyte population from a solid tumor or liquid tumor. The checkpoint inhibitor can an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. The agent that targets PD-1 can be an antibody or antibody format specific for PD-1, optionally selected from nivolumab, pembrolizumab, and pidilizumab. The agent that targets PD-L1 can an antibody or antibody format specific for PD-L1, optionally selected from atezolizumab, avelumab, durvalumab, and BMS-936559. The agent that targets CTLA-4 can be an antibody or antibody format specific for CTLA-4, optionally selected from ipilimumab and tremelimumab.

In some embodiments, the present disclosure provides a polynucleotide comprising a nucleic acid sequence encoding the antibody or antibody fragment. In some embodiments, a vector comprising the polynucleotide provided; in some embodiments, a host cell comprising the vector is provided.

In some aspects, the present disclosure provides a pharmaceutical composition comprising the antibody or antibody format of any of the antibodies disclosed herein and a pharmaceutically acceptable excipient.

The disclosure also provides kits that can simplify the evaluation of tumor or cancer cell specimens. A typical kit of the disclosure comprises various reagents including, for example, one or more agents (e.g., an antibody as disclosed herein) useful to detect a heterodimer. The kit can further comprise materials necessary for the evaluation, including welled plates, syringes, and the like. The kit can further comprise a label or printed instructions instructing the use of described reagents. The kit can further comprise a treatment to be tested.

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±15%, ±10%, ±5%, ±4%, ±3%, ±2%, ±1%, or ±less than 1%. Importantly, all recitations of a reference value preceded by the term “about” are intended to also be a recitation of the reference value alone. Notwithstanding the preceding, in this application the term “about” has a special meaning with regard to pharmacokinetic parameters, such as area under the curve (including AUC, AUCₜ, and AUC_{∞}) Cₘₐₓ, Tₘₐₓ, and the like. When used in relationship to a value for a pharmacokinetic parameter, the term “about” means from 85% to 115% of the reference parameter.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### Examples

FIG. 1 shows three line graphs of antigen reactivity of supernatants from three hybridomas “2H3”, “10C10” and “28A9” (see Table A), each expressing mouse IgG specific for the Bc12:Bim heterodimeric complex. This class of heterodimer specific Bc12:Bim reactive monoclonal antibodies are otherwise known as “HSB2B”. The supernatant concentrations were systematically reduced by serial dilution and subjected to enzyme linked immunosorbent assay (ELISA) on 96-well glutathione plates to confirm specific reactivity with heterodimer induced epitopes on Bc12, formed by complexing with Bim. As controls, reactivity with Bc1xL:Bim, Bc12 and Bc1xL monomers, and bovine serum albumin (BSA) were also screened against. To adhere the Bc1xL and Bc12 to the plates, each was expressed as a recombinant fusion protein with GST at the N-terminus (i.e. GST_Bc12 and GST_Bc1xL). Once adhered to the plate, incubations were either performed with a Bim peptide (which fits into the BH3-binding pocket of Bc12 family members), or PBS (thus retaining the monomeric confirmation). Hybridomas supernatants were subsequently incubated with the coated antigens and detected with HRP conjugated anti-mouse IgG. The reaction was developed with TMB substrate and stopped with sulphuric acid. Gluathione plate wells were scanned using a wavelength filter with an optical density of 450 nanometres (OD₄₅₀).

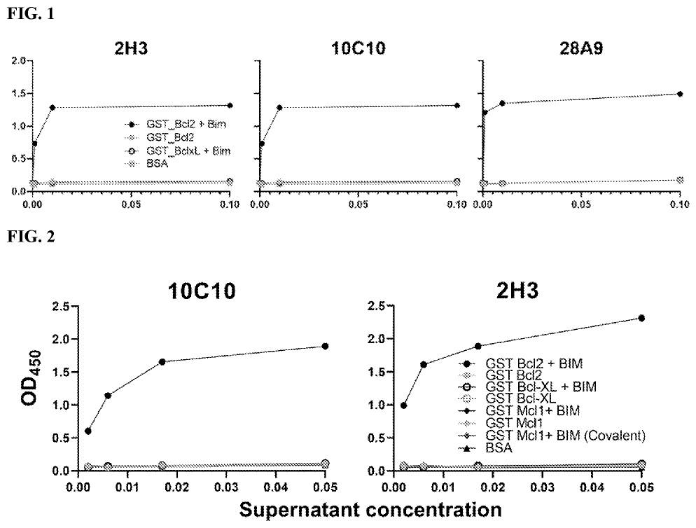

FIG. 2 is a display of two graphs analogous to those in FIG. 1, demonstrating HSB2B hybridoma supernatant (clones “10C10” and “2H3”, as indicated) specific reactivity for the Bc12:Bim complex, when tested amongst a more exhaustive range of antigens, including the Bc12 family member Mcl1. As a GST fusion protein (GST_Mc11), HSB2B hybridoma supernatant reactivity was tested against the Mc11 monomeric form, or the heterodimeric forms (created by, either, incubation of Bim peptide (as for FIG. 1), or by covalently linking the Bim peptide to the GST_Mc11). As for FIG. 1, the HSB2B mAbs show specific reactivity with the epitopes induced following the complexing of Bc12 with Bim.

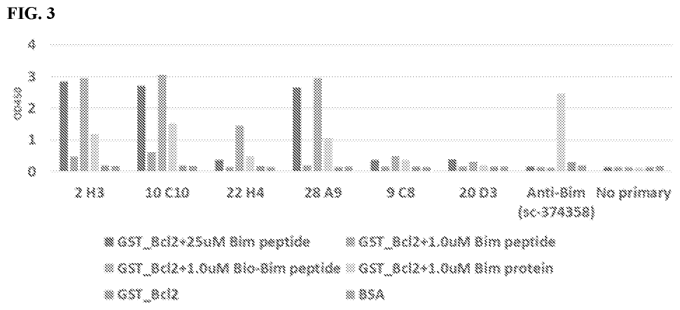

FIG. 3 is a bar graph demonstrating the capacity of the HSB2B mAbs to react with a physiologically relevant Bc12:Bim heterodimer complex formed using the full-length Bim protein. The experiment in FIG. 3 was performed to confirm the physiological relevance of the data produced in FIGS. 1-3 i.e. that the epitopes recognized by HSB2B mAbs are 1. formed when the complex is induced using the full-length Bim protein, and 2. not sterically hindered by the assembly the Bc12:Bim complex that contains the full-length protein. In analogous ELISA experiments to those outlined in FIGS. 1-2, HSB2B, clone supernatants from hybridomas “2H3”, “10C10”, “22H4”, “28A9”, “9C8” and “20D3” (see Table A) were diluted 1:10 and then screened for reactivity with the antigens listed beneath the graph. Following coating of GST_Bc12 to the glutathione plates, wells were either left as monomers or complexed with the Bim peptide (outlined in FIG. 1), a biotinylated variant of the Bim peptide (“Bio-Bim peptide”) or full-length recombinant “Bim protein”. The respective concentrations of the Bim variants are indicated. BSA coated wells served as negative controls. The commercially available mouse IgG anti-bim antibody (Santa-Cruz sc-374358) served as a positive control, confirming presence of the full-length bim protein. As a negative binding control, separate wells were only incubated with diluent, prior to the addition of the anti-mouse IgG HRP detection antibody and development.

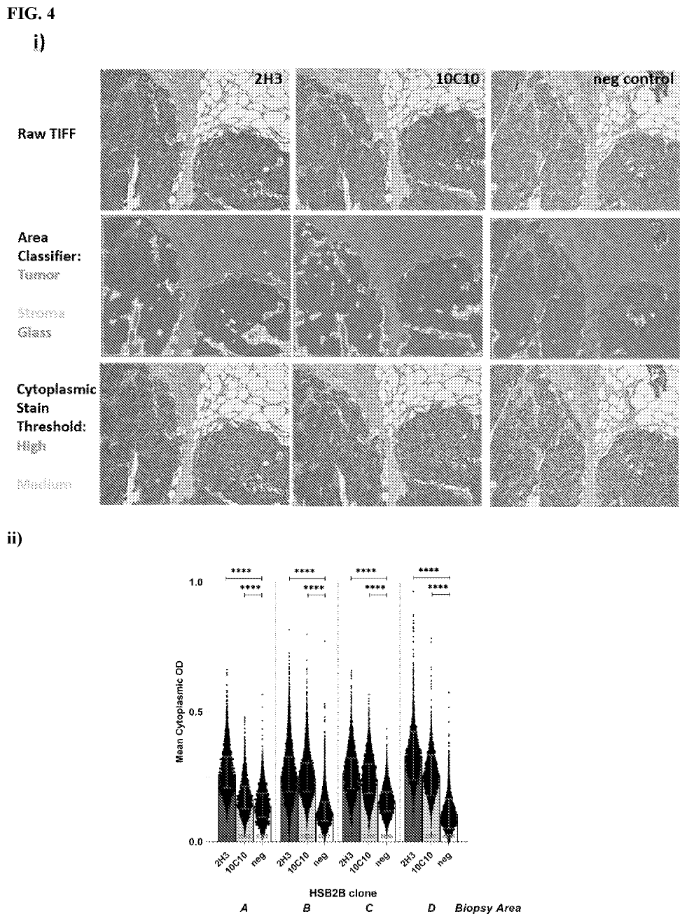

FIG. 4 demonstrates how, by immunohistochemistry (IHC) on fixed formalin paraffin embedded (FFPE) tissue, HSB2B clones “2H3” and “10C10” can react with small cell lung cancer tissue. Serial sections of a FFPE secondary (colon) tumor biopsy were stained with the HSB2B clones or, as a negative control, no primary antibody (“neg control”). Antibody binding was detected with anti-mouse IgG HRP and developed using a DAB substrate to yield a brown stain. Nuclei were counterstained blue with Hematoxylin. Four corresponding biopsy areas (A-D) were imaged per biopsy for analysis. FIG. 4 (i) is a panel of images showing the magnitude of staining with each HSB2B mAb for one of the four biopsy areas acquired for each stained biopsy. The raw TIFF image (top row) demonstrates the stained tissue. Cellular architecture (middle row) was then defined using HALO image analysis software (left hand color key, TIL=tumor infiltrating lymphocytes), most importantly to define the location of the tumor cells. A color code (left hand side) demonstrates the relative DAB (IgG binding) levels in the cytoplasm of individual tumor cells (defined and quantified using HALO), by using “low”, “medium” and “high” staining thresholds. Higher frequency of orange and red cells, when compared to the negative control, shows the higher frequency of highly stained cells by using the HSB2B mAbs. (ii) Dot plot/bar chart showing the cytoplasmic staining levels (Mean Cytoplasmic OD) for each of the tumor cells for the four different biopsy areas. The dots represent cytoplasmic staining intensities for individual cells. Medians and interquartile ranges are plotted. (Mann Whitney) statistical comparisons of staining intensity were performed between cells from HSB2B stained tissue and negative (neg) control tissue for each of the four areas of biopsy. ****p<0.001. There is a variance of tumor cell staining between biopsy areas (indicating a variance of bc12:bim expression in different areas of tumor), but, in each area of biopsy, higher level of cytoplasmic stain in the HSB2B stained versus unstained tumor cell cytoplasm.

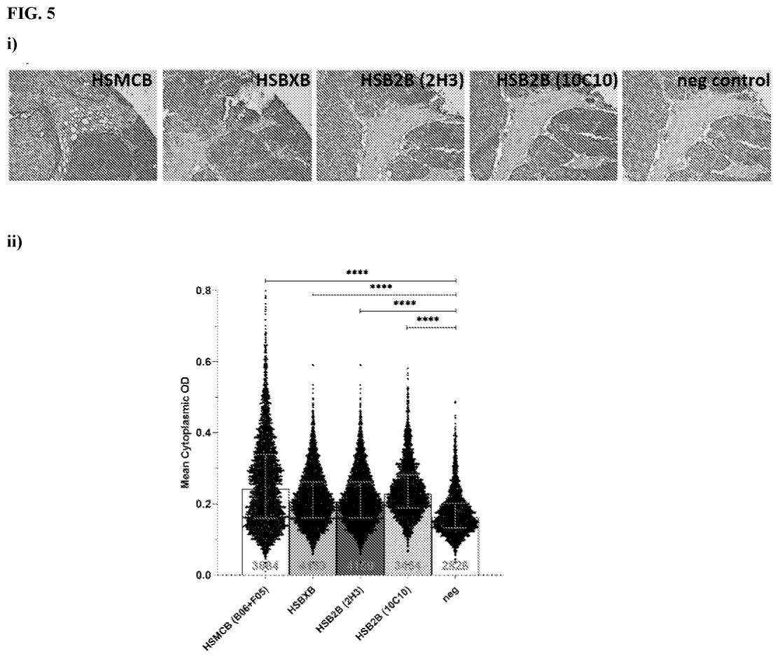

FIG. 5 shows IHC on FFPE small cell lung cancer tissue demonstrating that each of the present engineered heterodimer specific antibodies (HSAs) can have reactivity with small cell lung cancer. The present anti-BC12/BIM antibodies are labelled “HSB2B” and a clone number (see Table A); “HSBXB” is an antibody against Bc1-x1/Bim and “HSMCB” is an antibody against MCL-1/BIM. Using the staining method and matched patient sample to that described in FIG. 4, serial sections of small cell lung cancer biopsy were stained for relative reactivity with HSB2B mAbs 2H3 and 10C10, MC11:Bim specific mAb “HSMCB” and Bc1xL:Bim specific mAb “HSBXB.” Images containing color coded levels of cytoplasmic staining for individual cells from one representative biopsy area are demonstrated FIG. 5 (i), as performed fin FIG. 4 (i). FIG. 5 (ii) is cytoplasmic staining levels of individual cells from three different biopsy areas were combined and plotted as dot/bar graphs. The dots represent cytoplasmic staining intensities for individual cells. Medians and interquartile ranges are plotted. (Mann Whitney) statistical comparisons of staining intensity were performed between cells from HSA stained tissue and negative (neg) control tissue. **** p<0.001. There is a higher level of staining in the HSA stained versus unstained negative control tissue. The acquisition of data following staining with different HSA classes is planned to be of clinical utility to determine the relative dependencies of different cancers on different Bc12 family members.

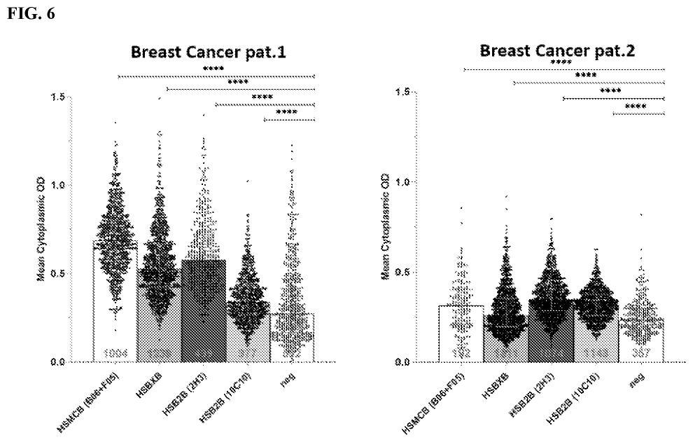

FIG. 6 is IHC on FFPE breast cancer tissue demonstrating that the present engineered HSAs can have different levels of reactivity with breast cancer biopsies from different patients. The present anti-BC12/BINI antibodies are labelled “HSB2B” and a clone number (see Table A); “HSBXB” is an antibody against Bc1-x1/Bim and “HSMCB” is an antibody against MCL-1/BIM. Using an analogous staining method to that described in FIG. 5, serial sections of two different patients' breast cancer biopsies (“Breast Cancer Patient 1 & 2”) were stained at the same time, under the identical conditions for relative reactivity with HSB2B mAbs 2H3 and 10C10, HSMCB and HSBXB. Cytoplasmic staining levels of individual cells from three different biopsy areas for each patient biopsy were combined and plotted as dot/bar graphs. There is a higher level of staining in the HSA stained versus unstained negative control tissue. The acquisition of data following staining with different HSA classes is planned to be of clinical utility to determine the relative dependencies of different cancers on different Bc12 family members. Between these two patients it can be appreciated that each patient has different absolute and relative levels of tumor cell reactivity with the different HSAs, suggestive of different orders of Bc12 family member dominance for survival between the two patients' tumors.

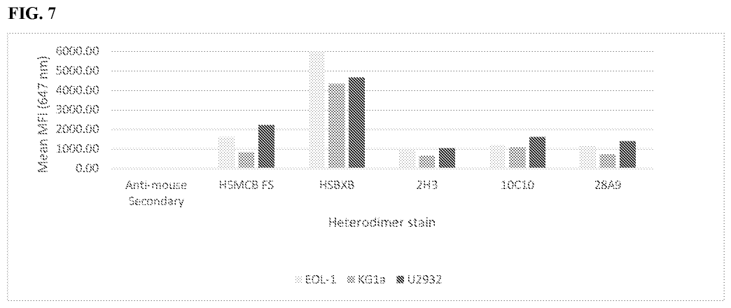

FIG. 7 is a bar graph demonstrating that each of the present HSAs can be used on flow cytometry. Cell-lines EOL-1, KG1a and U2932 (previously demonstrated to have high levels of the Bc12:Bim heterodimer) were stained with the indicated HSAs and detected with an Alexa Fluor 647 conjugated anti-mouse IgG. The present anti-BC12/BINI antibodies are labelled “HSB2B” and a clone number (see Table A); “HSBXB” is an antibody against Bc1-x1/Bim and “HSMCB” is an antibody against MCL-1/BIM. A negative control set of cells was not stained with a primary antibody (“Anti-mouse secondary”). The median relative levels of reactivity and standard deviations are plotted for each of the stained populations. The signal was greater when the HSAs were added to the cells (above that of the negative control), confirming reactivity of the HSAs to the different cells. Importantly each of the cell-lines had different levels of reactivity with each of the HSAs. As planned for IHC, clinically useful companion diagnostic decision making is foreseen by appreciating the relative and absolute levels of reactivity of liquid biopsies with these different antibodies.

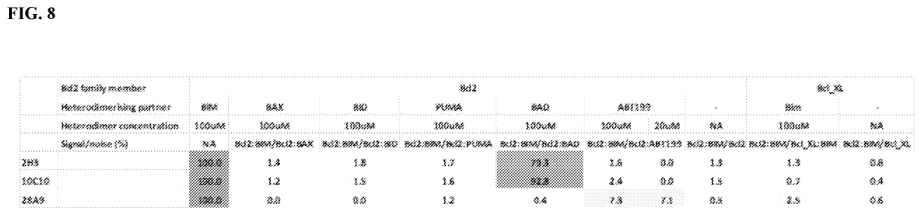

FIG. 8 shows HSB2B reactivity with different Bc12 and Bc1_XL heterodimers. ELISA data demonstrating the reactivities of respective HSB2B mAb clones for different Bc12: BIM and Bc1_XL heterodimers and monomers are shown. All levels of reactivity are tabulated as percentage magnitudes of signal relative to those yielded with the Bc12:BIM heterodimer. The reactivities with the Bc12 inhibitor ABT199 in a heterodimer with Bc12 are also demonstrated. Clones 2H3 and 10C10 have cross-reactivity between Bc12:BIM and Bc12:BAX heterodimers, whereas clone 28A9 physiological heterodimer reactivity is restricted to Bc12:BIM. 28A9 has low ABT199 cross-reactivity.

What is claimed is:

1. A composition comprising an antibody or antibody format, or fragment thereof, comprising:
(A) (i) a heavy chain variable region comprising heavy chain CDR1, CDR2, and CDR3 sequences, wherein the heavy chain CDR1 sequence is GYTFTSYW (SEQ ID NO: 2), or a variant thereof, the heavy chain CDR2 sequence is IDPNSGGT (SEQ ID NO: 3), or a variant thereof, and the heavy chain CDR3 sequence is ARWWDYDYFDY (SEQ ID NO: 4), or a variant thereof, and (ii) a light chain variable region comprising light chain CDR1, CDR2, and CDR3 sequences, wherein the light chain CDR1 sequence is QSLLNSGNQKNY (SEQ ID NO: 6), or a variant thereof, the light chain CDR2 sequence is WAS (SEQ ID NO: 7), or a variant thereof, and the light chain CDR3 sequence is QNDYNYPLT (SEQ ID NO: 8), or a variant thereof.

2. The composition of claim 1, wherein the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

3. The composition of claim 2, wherein the variable region FW sequences are human.

4. The composition of any one of claims 1-3, wherein the antibody or antibody format, or fragment thereof, comprises a human heavy chain and light chain constant regions.

5. The composition of any one of claims 1-4, wherein the constant regions are selected from the group consisting of human IgG1, IgG2, IgG3, and IgG4.

6. The composition of any one of claims 1-5, wherein the antibody or antibody format, or fragment thereof, comprises:
(A) (i) a heavy chain variable region sequence comprising the amino acid sequence set forth in SEQ ID NO: 1, or the amino acid sequence of SEQ ID NO: 1 having at least about 90% identity thereto; and (ii) a light chain variable region sequence comprising the amino acid sequence of SEQ ID NO: 5, or the amino acid sequence of SEQ ID NO: 5 having at least about 90% identity thereto.

7. The composition of claim 6, wherein the antibody or antibody format, or fragment thereof, comprises an amino acid sequence having at least about 95%, or 97%, or 98% identity with SEQ ID NO: 1 and/or SEQ ID NO. 5.

8. A polynucleotide comprising a nucleic acid sequence encoding the antibody or antibody format, or fragment thereof of any one of claims 1-7.

9. A vector comprising the polynucleotide of claim 8.

10. A host cell comprising the vector of claim 9.

11. A pharmaceutical composition comprising the antibody or antibody format, or fragment thereof, of any one of claims 1-7, and a pharmaceutically acceptable excipient.

12. A composition comprising an antibody or antibody format, or fragment thereof comprising:
(A) (i) a heavy chain variable region comprising heavy chain CDR1, CDR2, and CDR3 sequences, wherein the heavy chain CDR1 sequence is GYSITSGYY (SEQ ID NO: 18), or a variant thereof, the heavy chain CDR2 sequence is ISYDGSN (SEQ ID NO: 19), or a variant thereof, and the heavy chain CDR3 sequence is ARDGTTVVATLAY (SEQ ID NO: 20), or a variant thereof, and (ii) a light chain variable region comprising light chain CDR1, CDR2, and CDR3 sequences, wherein the light chain CDR1 sequence is SSVSY (SEQ ID NO: 22), or a variant thereof, the light chain CDR2 sequence is RTS (SEQ ID NO: 23), or a variant thereof, and the light chain CDR3 sequence is QQYHSYPPT (SEQ ID NO: 24), or a variant thereof or
(B) (i) a heavy chain variable region comprising heavy chain CDR1, CDR2, and CDR3 sequences, wherein the heavy chain CDR1 sequence is GYTFASYW(SEQ ID NO: 26), or a variant thereof, the heavy chain CDR2 sequence is IDPNSGGT (SEQ ID NO: 27), or a variant thereof, and the heavy chain CDR3 sequence is ARWWDYDYFDY (SEQ ID NO: 28), or a variant thereof, and (ii) a light chain variable region comprising light chain CDR1, CDR2, and CDR3 sequences, wherein the light chain CDR1 sequence is QSLLNSGNQKRY (SEQ ID NO: 30), or a variant thereof, the light chain CDR2 sequence is WAS (SEQ ID NO: 31), or a variant thereof, and the light chain CDR3 sequence is QNDYNYPLT (SEQ ID NO: 32), or a variant thereof.

13. The composition of claim 12, wherein the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

14. The composition of claim 13, wherein the variable region FW sequences are human.

15. The composition of any one of claims 12-14, wherein the antibody or antibody format, or fragment thereof, comprises a human heavy chain and light chain constant regions.

16. The composition of any one of claims 12-15, wherein the constant regions are selected from the group consisting of human IgG1, IgG2, IgG3, and IgG4.

17. The composition of any one of claims 12-16, wherein the antibody or antibody format, or fragment thereof, comprises:
(A) (i) a heavy chain variable region sequence comprising the amino acid sequence set forth in SEQ ID NO: 17, or the amino acid sequence of SEQ ID NO: 17 having at least about 90% identity thereto; and (ii) a light chain variable region sequence comprising the amino acid sequence of SEQ ID NO: 21, or the amino acid sequence of SEQ ID NO: 21 having at least about 90% identity thereto or
(B) (i) a heavy chain variable region sequence comprising the amino acid sequence set forth in SEQ ID NO: 25, or the amino acid sequence of SEQ ID NO: 25 having at least about 90% identity thereto; and (ii) a light chain variable region sequence comprising the amino acid sequence of SEQ ID NO: 29, or the amino acid sequence of SEQ ID NO: 29 having at least about 90% identity thereto.

18. The composition of claim 17, wherein the antibody or antibody format, or fragment thereof, comprises an amino acid sequence having at least about 95%, or 97%, or 98% identity with SEQ ID NO: 17 and/or SEQ ID NO. 21 of SEQ ID NO: 25 and/or SEQ ID NO: 29.

19. A polynucleotide comprising a nucleic acid sequence encoding the antibody or antibody format, or fragment thereof of any one of claims 12-18.

20. A vector comprising the polynucleotide of claim 19.

21. A host cell comprising the vector of claim 20.

22. A pharmaceutical composition comprising the antibody or antibody format, or fragment thereof, of any one of claims 12-18, and a pharmaceutically acceptable excipient.

23. A method for predicting a patient's sensitivity or response to a cancer treatment, comprising:
(a) contacting a sample with an antibody or antibody format, or fragment thereof, from any one of claims 1-18, wherein the antibody recognizes a heterodimer comprising two B-cell lymphoma 2 (BCL-2) proteins selected from Bc12 and BIM, the sample being a specimen from a solid tumor or liquid tumor of the patient;
(b) detecting a signal that indicates the amount of the heterodimer; and
(c) determining a ratio of the amount of heterodimer present in the sample from step (b) to a reference value,
wherein the reference value comprises the amount of one of the Bc12 and BIM monomers of the heterodimer in the sample,
the ratio being predictive of the patient's sensitivity to the cancer treatment.

24. A method for predicting a patient's sensitivity or response to a cancer treatment, comprising:
(a) contacting a sample with an antibody or antibody format, or fragment thereof, from any one of claims 1-18, wherein the antibody recognizes a heterodimer comprising two B-cell lymphoma 2 (BCL-2) proteins selected from Bc12 and BIM, and an antibody or antibody format, or fragment thereof, that recognizes one of the Bc12 and BIM protein monomers of the heterodimer, the sample being a specimen from a solid tumor or liquid tumor of the patient;
(b) detecting a signal that indicates the amount of the heterodimer and a signal that indicates the amount of the monomer; and
(c) determining a ratio based on the amount heterodimer to the amount of the monomer, the ratio being predictive of the patient's sensitivity to the cancer treatment.

25. The method of claim 23 or 24, further comprising administering a cancer treatment to the patient if the ratio is predictive of sensitivity to the cancer treatment.

26. The method of claim 25, further comprising treating the patient with a reduced dose or less frequent and/or shortened regimen of the cancer treatment if the ratio is predictive of sensitivity to the cancer treatment.

27. The method of claim 25, further comprising treating the patient with an increased dose or more frequent and/or prolonged regimen of the cancer treatment if the ratio is predictive of a lack of sensitivity to the cancer treatment.

28. The method of claim 23 or 24, further comprising withholding cancer treatment from the patient if the ratio is predictive of a lack of sensitivity to the cancer treatment.

29. The method of claim 23 or 24, further comprising treating the patient with a different cancer treatment if the ratio is predictive of a lack of sensitivity to the cancer treatment.

30. The method of any one of claims 23-29, further comprising determining one or more clinical factors of the patient.

31. The method of claim 30, further comprising classifying the patient for likelihood of clinical response to the cancer treatment based on one or more clinical factors of the patient.

32. The method of claim 31, further comprising comparing the prediction of the patient's sensitivity to the cancer treatment with the likelihood of clinical response to the cancer treatment based on one or more clinical factors of the patient.

33. The method of any one of claims 30-32, wherein the clinical factor is one or more of age, cytogenetic status, performance, histological subclass, gender, and disease stage.

34. The method of any one of claims 23-32, further comprising measuring an additional biomarker selected from mutational status, single nucleotide polymorphisms, steady state protein levels, and dynamic protein levels.

35. The method of any one of claims 23-34, wherein the detection of the heterodimer employs an immunohistochemistry (IHC), flow cytometry, or immunofluorescent method.

36. The method of any one of claims 23-35, wherein the method provides a ratio of the Bc12/BIM heterodimer to one of Bc12 and BIM monomer.

37. The method of any one of claims 23-36, wherein the cancer treatment comprises a BH3 mimetic.

38. The method of claim 37, wherein the BH3 mimetic is selected from ABT-737 and ABT-263 (navitoclax), Venetoclax (Venclexta, ABT-199), 563845, AMG176, ADZ5991, A-1155463, A1331852, EU5346, or combinations thereof.

39. The method of any one of claims 23-38, wherein the cancer treatment comprises one or more chemotherapy agents.

40. The method of any one of claims 23-39, wherein the cancer treatment is one or more of a SMAC mimetic, proteasome inhibitor, histone deacetylase inhibitor, glucocorticoid, steroid, monoclonal antibody, antibody-drug conjugate, or thalidomide derivative.

41. The method of any one of claims 23-40, wherein the cancer treatment blocks formation of the particular heterodimer detected.

42. The method of any one of claims 23-40, wherein the cancer treatment perturbs or reduces formation of the detected heterodimer.

43. The method of any one of claims 23-36, wherein the cancer treatment comprises a checkpoint inhibitor.

44. The method of claim 43, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

45. The method of claim 44, wherein the agent that targets PD-1 is an antibody or antibody format specific for PD-1, optionally selected from nivolumab, pembrolizumab, and pidilizumab.

46. The method of claim 44, wherein the agent that targets PD-L1 is an antibody or antibody format specific for PD-L1, optionally selected from atezolizumab, avelumab, durvalumab, and BMS-936559.

47. The method of claim 44, wherein the agent that targets CTLA-4 is an antibody or antibody format specific for CTLA-4, optionally selected from ipilimumab and tremelimumab.

48. The method of any one of claims 23-47, wherein the sample is selected from a tumor biopsy, tissue biopsy, tumor resection, frozen tumor tissue specimen, lymph node, bone marrow, circulating tumor cells, cultured cells, a formalin-fixed paraffin embedded tumor tissue specimen, bronchoalveolar lavage, skin, hair, urine, and combinations thereof.

49. The method of claim 48, wherein the tumor biopsy is selected from a core biopsy, needle biopsy, surgical biopsy, and an excisional biopsy.

50. The method of any one of claims 23-47, wherein the sample is an infiltrating lymphocyte of the patient.

51. The method of any one of claims 23-47, wherein the solid tumor is selected from lung cancer, breast cancer, prostate cancer, melanoma, pancreatic cancer, kidney cancer, colon cancer, and ovarian cancer.

52. The method of claim 51, wherein the lung cancer is selected from non-small cell lung cancer (NSCLC) and small cell lung cancer (SCLC).

53. The method of claim 51, wherein the breast cancer is triple negative breast cancer.

54. The method of claim 51, wherein the prostate cancer is androgen independent prostate cancer.

55. The method of claim 23, wherein the sensitivity is characterized by a higher likelihood for response to the cancer treatment.

56. The method of any one of claims 23-55, wherein the method does not involve a functional readout of mitochondrial outer membrane permeabilization (MOMP).

57. The method of any one of claims 23-55, wherein the method does not involve a dye-based detection of cell membrane potential.

58. The method of any one of claims 23-57, wherein the antibody or antibody format, or fragment thereof is selected from one or more of a monoclonal antibody, polyclonal antibody, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody.

59. The method of any one of claims 23-58, wherein the likelihood of clinical response is defined by the following equation:
$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + … / ( n peptides )$$
wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

60. The method of any one of claims 23-58, wherein the likelihood of clinical response is defined by the following equation:
$$\% Priming = [ 100 * ( \frac{{DMSO}_{a v g} A UC - {Peptide}_{n} A U C}{{DMSO}_{a v g} A U C - C C C {P}_{a v g} A U C} ) ]$$
wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

61. The method of claim 59 or 60, wherein the one or more clinical factors are selected to increase specificity and/or sensitivity of the BH3 profile for association with clinical response.

62. A method for predicting a patient's responsiveness to a checkpoint inhibitor in a sample, comprising measuring the amount of an antibody comprising a Bc12/BIM heterodimer selected from any one of claims 1-18, wherein the sample comprises an infiltrating lymphocyte population from a solid tumor or liquid tumor.

63. The method of claim 62, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD 160, TIGIT, SIRPα, ICOS, CD172a and TMIGD2.

64. The method of claim 63, wherein the agent that targets PD-1 is an antibody or antibody format specific for PD-1, optionally selected from nivolumab, pembrolizumab, and pidilizumab.

65. The method of claim 63 or 64, wherein the agent that targets PD-L1 is an antibody or antibody format specific for PD-L1, optionally selected from atezolizumab, avelumab, durvalumab, and BMS-936559.

66. The method of claim 63, wherein the agent that targets CTLA-4 is an antibody or antibody format specific for CTLA-4, optionally selected from ipilimumab and tremelimumab.

67. A method of generating a Bc12 and BIM heterodimer antibody, comprising:
(a) immunizing a subject with a heterodimer induced conformation antigen;
(b) isolating from the subject a splenic B cell producing the IgG recognizing the heterodimer induced antigen;
(c) passing the splenic B cell onto a magnetic column for negative selection, wherein the magnetic column for negative selection is coated with a recombinant fusion protein containing one monomer of the heterodimer;
(d) collecting the flow through of the splenic B cells from the magnetic column for negative selection, and passing the flow through onto a magnetic column for positive selection; wherein the magnetic column for positive selection is coated with the heterodimer antigen;
(e) eluting and collecting the splenic B cells bound to the magnetic column for positive selection;
(f) culturing the collected cells in a B-cell media; and
(g) isolating the heterodimer specific antibody from the cultured cells, thereby generating a heterodimer antibody.

68. The method of claim 67, wherein the subject is a human, a monkey, a mouse, a rat, or a hamster.
